SEQUENCE LISTING (2 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 156 bp, Homo sapiens
  source 1..156: mol_type = genomic DNA
tataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacata

(19) United States
(12) Patent Application Publication
(10) Pub. No.: US 2026/0199451 A1
(43) Pub. Date: Jul. 16, 2026

(54) VACCINE COMPOSITIONS AND METHODS

(71) Applicant: FLAGSHIP PIONEERING INNOVATIONS VII, LLC, Cambridge, MA (US)

(72) Inventors: Jacob Rosenblum Rubens, Cambridge, MA (US); Louisa Marie Helms, Seattle, WA (US); Max Everett Distler, Allston, MA (US); Edward Matthew Kennedy, Bedford, MA (US)

(21) Appl. No.: 19/443,595

(22) Filed: Jan. 8, 2026

Related U.S. Application Data
(60) Provisional application No. 63/743,950, filed on Jan. 10, 2025.
(60) Provisional application No. 63/800,782, filed on May 6, 2025.
(60) Provisional application No. 63/945,048, filed on Dec. 19, 2025.

(51) Int. Cl. A61K 39/215; A61K 9/1272; A61K 9/51; A61K 39/00; A61P 31/14; C12N 7/00; C12N 15/113

(52) U.S. Cl. CPC A61K 39/215; A61K 9/1272; A61K 9/5123; A61P 31/14; C12N 7/00; C12N 15/113; A61K 2039/53; A61K 2039/545; A61K 2039/55555; A61K 2039/572; A61K 2039/575; C12N 2310/11; C12N 2310/335; C12N 2770/20034; C12N 2830/50

(57) ABSTRACT

The present disclosure provides, for example, vaccine compositions comprising an mRNA molecule and a double stranded DNA molecule. The DNA molecule may be hemi-modified. The mRNA molecule and DNA molecule may encode the same or similar immunogen. In some embodiments, the combination of the mRNA molecule and the DNA molecule evokes a stronger immune response than either one alone. Methods of making and using the vaccine compositions are also described.

### RELATED APPLICATIONS

This application claims priority to U.S. Ser. No. 63/743,950, filed on Jan. 10, 2025, U.S. Ser. No. 63/800,782, filed on May 6, 2025, and U.S. Ser. No. 63/945,048, filed on Dec. 19, 2025, the entire contents of each of which are incorporated herein by reference.

### SEQUENCE LISTING

The instant application contains a Sequence Listing which has been submitted electronically in XML format and is hereby incorporated by reference in its entirety. Said XML copy, created on Dec. 30, 2025, is named F2128-703110_SL.xml and is 4,019 bytes in size.

### BACKGROUND

There is a need for novel therapeutic modalities to address unmet medical need.

### SUMMARY OF THE INVENTION

Described herein are pharmaceutical compositions, constructs, preparations, methods of using such compositions, constructs and preparations, and methods of making the same.

### ENUMERATED EMBODIMENTS

1. A pharmaceutical composition comprising:
(a) an mRNA molecule encoding a first immunogen; and
(b) a double stranded DNA molecule encoding: (i) the first immunogen, (ii) a second immunogen having at least 90%, at least 95%, at least 98%, or at least 99% amino acid sequence identity to the first immunogen, or (iii) a second immunogen that is recognized by an antigen binding site of an antibody or T cell receptor (TCR), wherein the antigen binding site also recognizes the first immunogen.
   2. A method of inducing an immune response in a subject, the method comprising administering to the subject the pharmaceutical composition of embodiment 1.
   3. A method of inducing an immune response in a subject, the method comprising administering to the subject:
(a) a composition comprising an mRNA molecule encoding a first immunogen; and
(b) a composition comprising a double stranded DNA molecule encoding: (i) the first immunogen, (ii) a second immunogen having at least 90%, at least 95%, at least 98%, or at least 99% amino acid sequence identity to the first immunogen, or (iii) a second immunogen that is recognized by an antigen binding site of an antibody or TCR, wherein the antigen binding site also recognizes the first immunogen.
   4. The method of embodiment 2 or 3, wherein (a) and (b) are administered simultaneously or sequentially.
   5. The method of any of embodiments 2-4, wherein (a) and (b) are admixed or are not admixed.
   6. The method of any one of embodiments 2-5, wherein (a) and (b) are admixed and are administered as a single dose.
   7. The method of any one of embodiments 2-5, wherein (a) and (b) are admixed and are administered as 2 or more doses.
   8. The method of any one of embodiments 2-5, wherein (a) is administered as 2 or more doses.
   9. The method of any one of embodiments 2-5, wherein (b) is administered as 2 or more doses.
   10. A kit comprising:
(a) an mRNA molecule encoding a first immunogen; and
(b) a double stranded DNA molecule encoding: (i) the first immunogen, (ii) a second immunogen having at least 90%, at least 95%, at least 98%, or at least 99% amino acid sequence identity to the immunogen, or (iii) a second immunogen that is recognized by an antigen binding site of an antibody or TCR, wherein the antigen binding site also recognizes the first immunogen.
   11. The kit of embodiment 10, wherein (a) is situated in a first container and (b) is situated in a second container.
   12. The pharmaceutical composition, method, or kit of any one of the preceding embodiments, wherein the first immunogen is a viral antigen, a bacterial antigen, a fungal antigen, or a tumor antigen.
   13. The pharmaceutical composition, method, or kit of embodiment 12, wherein the viral antigen is a COVID-19 antigen, a hepatitis antigen (e.g., a hepatitis B antigen or a hepatitis A antigen), an influenza antigen, a human papillomavirus (HPV) antigen, a chickenpox antigen, a measles antigen, a mumps antigen, a rubella antigen, or a combination thereof.
   14. The pharmaceutical composition, method, or kit of embodiment 12, wherein the bacterial antigen is a diphtheria antigen, a tetanus antigen, a pertussis antigen, a meningitidis antigen, a tuberculosis antigen, a cholerae antigen, or a combination thereof.
   15. The pharmaceutical composition, method, or kit of any one of the preceding embodiments, wherein the first immunogen is a peptide.
   16. The pharmaceutical composition, method, or kit of any one of the preceding embodiments, wherein the first immunogen has a length of 10-20, 20-50, 50-100, 100-500, 500-1000, 1000-2000, or 2000-5000 amino acids.
   17. The pharmaceutical composition, method, or kit of any one of the preceding embodiments, wherein (b) comprises the double stranded DNA molecule encoding (i) the first immunogen.
   18. The pharmaceutical composition, method, or kit of any one of the preceding embodiments, wherein (b) comprises the double stranded DNA molecule encoding (ii) the second immunogen having at least 90%, at least 95%, at least 98%, or at least 99% amino acid sequence identity to the first immunogen.
   19. The pharmaceutical composition, method, or kit of any one of the preceding embodiments, wherein (b) comprises the double stranded DNA molecule encoding (iii) the second immunogen that is recognized by an antigen binding site of an antibody or T cell receptor (TCR), wherein the antigen binding site also recognizes the first immunogen.
   20. The pharmaceutical composition, method, or kit of any one of the preceding embodiments, wherein the first immunogen is not a pneumococcal polypeptide antigen.
   21. The pharmaceutical composition, method, or kit of any one of the preceding embodiments, wherein the first immunogen is not a Bacilli antigen, not a Lactobacillies antigen, or not a Streptococcaceae antigen.
   22. The pharmaceutical composition, method, or kit of any one of the preceding embodiments, wherein the first immunogen is not a COVID antigen.
   23. The pharmaceutical composition, method, or kit of any one of the preceding embodiments, wherein the first immunogen is not a coronavirus antigen.
   24. The pharmaceutical composition, method, or kit of any one of the preceding embodiments, wherein the first immunogen is not an HIV antigen.
   25. The pharmaceutical composition, method, or kit of any one of the preceding embodiments, wherein the first immunogen is not a lentivirus antigen.
   26. The pharmaceutical composition, method, or kit of any one of the preceding embodiments, wherein the second immunogen is not a pneumococcal polypeptide antigen.
   27. The pharmaceutical composition, method, or kit of any one of the preceding embodiments, wherein the second immunogen is not a Bacilli antigen, not a Lactobacillies antigen, or not a Streptococcaceae antigen.
   28. The pharmaceutical composition, method, or kit of any one of the preceding embodiments, wherein the second immunogen is not a COVID antigen.
   29. The pharmaceutical composition, method, or kit of any one of the preceding embodiments, wherein the second immunogen is not a coronavirus antigen.
   30. The pharmaceutical composition, method, or kit of any one of the preceding embodiments, wherein the second immunogen is not an HIV antigen.
   31. The pharmaceutical composition, method, or kit of any one of the preceding embodiments, wherein the second immunogen is not a lentivirus antigen.
   32. The pharmaceutical composition, method, or kit of any one of the preceding embodiments, wherein the mRNA molecule comprises a 5′ cap.
   33. The pharmaceutical composition, method, or kit of any one of the preceding embodiments, wherein the mRNA molecule comprises a polyadenylation (polyA) tail.
   34. The pharmaceutical composition, method, or kit of any one of the preceding embodiments, wherein the mRNA molecule comprises a 5′ untranslated region (UTR).
   35. The pharmaceutical composition, method, or kit of any one of the preceding embodiments, wherein the mRNA molecule comprises a 3′ UTR.
   36. The pharmaceutical composition, method, or kit of any one of the preceding embodiments, wherein the mRNA molecule comprises one or more chemically modified ribonucleotides.
   37. The pharmaceutical composition, method, or kit of embodiment 36, wherein the chemically modified ribonucleotide comprises 5-methoxyuracil.
   38. The pharmaceutical composition, method, or kit of any one of the preceding embodiments, wherein the mRNA molecule further comprises one or more (e.g., 2 or all) of: (i) 5′ UTR; (ii) 3′ UTR; and (iii) polyA tail.
   39. The pharmaceutical composition, method, or kit of any of the preceding embodiments, wherein the mRNA molecule is circular or linear.
   40. The pharmaceutical composition, method, or kit of any of the preceding embodiments, wherein the DNA molecule is circular or linear.
   41. The pharmaceutical composition, method, or kit of any one of the preceding embodiments, wherein the DNA molecule comprises a plasmid or a minicircle.
   42. The pharmaceutical composition, method, or kit of any of the preceding embodiments, wherein the DNA molecule lacks a material portion of vector backbone (e.g., plasmid backbone).
   43. The pharmaceutical composition, method, or kit of any of the preceding embodiments, wherein the DNA molecule does not comprise a bacterial origin of replication.
   44. The pharmaceutical composition, method, or kit of any of the preceding embodiments, wherein the DNA molecule does not comprise an antibiotic resistance selectable marker.
   45. The pharmaceutical composition, method, or kit of any of the preceding embodiments, wherein the DNA molecule is not supercoiled.
   46. The pharmaceutical composition, method, or kit of any of the preceding embodiments, wherein the DNA molecule comprises:
(i) an upstream DNA end form which is a closed end;
(ii) a double stranded region; and
(iii) a downstream DNA end form which is a closed end.
   47. The pharmaceutical composition, method, or kit of any of the preceding embodiments, wherein the DNA molecule is resistant to endonuclease digestion.
   48. The pharmaceutical composition, method, or kit of any one of the preceding embodiments, wherein the DNA molecule comprises one or more chemically modified nucleotides.
   49. The pharmaceutical composition, method, or kit of any one of the preceding embodiments, wherein the DNA molecule comprises a promoter sequence operably linked to the sequence encoding the first immunogen or second immunogen.
   50. The pharmaceutical composition, method, or kit of any one of the preceding embodiments, wherein at least 80%, at least 85%, at least 90%, at least 95%, at least 98%, at least 99%, or 100% of the sugars of the DNA molecule are deoxyribose sugars.
   51. The pharmaceutical composition, method, or kit of any one of the preceding embodiments, wherein all positions in the DNA molecule comprise a deoxyribose sugar.
   52. The pharmaceutical composition, method, or kit of any one of the preceding embodiments, wherein the DNA molecule further comprises one or more of (e.g., 2, 3, 4, or all of): (i) a sequence encoding a 5′ UTR; (ii) a sequence encoding a 3′ UTR, (iii) a Woodchuck Hepatitis Virus Posttranscriptional Regulatory Element (WPRE); (iv) an intron sequence; or (v) an enhancer sequence.
   53. The pharmaceutical composition, method, or kit of any one of the preceding embodiments, wherein the mRNA molecule and DNA molecule are present at a molar ratio of between 10:1 and 1:10 mRNA:DNA.
   54. The pharmaceutical composition, method, or kit of any one of the preceding embodiments, wherein the mRNA molecule and DNA molecule are present at a molar ratio of between 5:1 and 1:5 mRNA:DNA.
   55. The pharmaceutical composition, method, or kit of any one of the preceding embodiments, wherein the mRNA molecule and DNA molecule are present at a molar ratio of between 2:1 and 1:2 mRNA:DNA.
   56. The pharmaceutical composition, method, or kit of any one of the preceding embodiments, wherein the mRNA molecule and DNA molecule are present at a molar ratio of about 1:1 mRNA:DNA.
   57. The pharmaceutical composition, method, or kit of any one of the preceding embodiments, wherein the mRNA molecule is present at a higher molar concentration than the DNA molecule.
   58. The pharmaceutical composition, method, or kit of any one of embodiments 1-56, wherein the mRNA molecule is present at a lower molar concentration than the DNA molecule.
   59. The pharmaceutical composition, method, or kit of any one of the preceding embodiments, wherein the mRNA molecule comprises a first immunogen sequence encoding the first immunogen and the DNA molecule comprises a second immunogen sequence encoding the second immunogen, and wherein the first immunogen sequence and second immunogen sequence have identical nucleic acid sequences.
   60. The pharmaceutical composition, method, or kit of any one of the preceding embodiments, wherein the mRNA molecule comprises a first immunogen sequence encoding the first immunogen and the DNA molecule comprises a second immunogen sequence encoding the second immunogen, and wherein the first immunogen sequence and second immunogen sequence have identical nucleic acid sequences, except that the DNA molecule comprises one or more introns that are absent from the mRNA molecule.
   61. The pharmaceutical composition, method, or kit of any one of embodiments 1-58, wherein the mRNA molecule comprises a first immunogen sequence encoding the first immunogen and the DNA molecule comprises a second immunogen sequence encoding the second immunogen, and wherein the first immunogen sequence and second immunogen sequence have sequences that differ at one or more nucleotides.
   62. The pharmaceutical composition, method, or kit of any one of embodiments 1-58 or 61, wherein the mRNA molecule comprises a first immunogen sequence encoding the first immunogen and the DNA molecule comprises a second immunogen sequence encoding the second immunogen, and wherein the first immunogen sequence and second immunogen sequence are at least 90%, at least 95%, at least 98%, or at least 99% identical.
   63. The pharmaceutical composition, method, or kit of any one of embodiments 1-58, 61, or 62, wherein the mRNA molecule comprises a first immunogen sequence encoding the first immunogen and the DNA molecule comprises a second immunogen sequence encoding the second immunogen, and wherein the first immunogen sequence and second immunogen sequence are 90%-95%, 95%-98%, or 98%-99% identical.
   64. The pharmaceutical composition, method, or kit of any one of the preceding embodiments, wherein the composition (e.g., the pharmaceutical composition or the composition of (a) and/or (b)) or kit is substantially free of (e.g., is free of) protein.
   65. The pharmaceutical composition, method, or kit of any one of the preceding embodiments, wherein the composition (e.g., the pharmaceutical composition or the composition of (a) and/or (b)) or kit is substantially free of (e.g., is free of) virus.
   66. The pharmaceutical composition, method, or kit of any of one of the preceding embodiments, wherein the composition (e.g., the pharmaceutical composition or the composition of (a) and/or (b)) or kit is substantially free of (e.g., is free of) viral protein.
   67. The pharmaceutical composition, method, or kit of any one of the preceding embodiments, wherein the composition (e.g., the pharmaceutical composition or the composition of (a) and/or (b)) or kit is substantially free of (e.g., is free of) bacteria.
   68. The pharmaceutical composition, method, or kit of any one of the preceding embodiments, wherein the composition (e.g., the pharmaceutical composition or the composition of (a) and/or (b)) or kit comprises a pharmaceutically acceptable excipient.
   69. The pharmaceutical composition, method, or kit of any one of the preceding embodiments, wherein the composition (e.g., the pharmaceutical composition or the composition of (a) and/or (b)) or kit comprises a pharmaceutically acceptable diluent.
   70. The pharmaceutical composition, method, or kit of any one of the preceding embodiments, wherein the composition (e.g., the pharmaceutical composition or the composition of (a) and/or (b)) or kit comprises a pharmaceutically acceptable adjuvant.
   71. The pharmaceutical composition, method, or kit of any one of the preceding embodiments, wherein the composition or kit further comprises a lipid nanoparticle (LNP). 72. The pharmaceutical composition, method, or kit of any one of the preceding embodiments, wherein the composition or kit comprises an LNP that comprises the mRNA molecule and the DNA molecule.
   73. The pharmaceutical composition, method, or kit of any one of the preceding embodiments, which comprises:
(i) a first LNP that comprises the mRNA molecule, and
(ii) a second LNP that comprises the DNA molecule.
   74. The pharmaceutical composition, method, or kit of any one of embodiments 71-73, wherein the LNP comprises a cationic lipid (e.g., an ionizable lipid), a non-cationic lipid (e.g., phospholipid), a structural lipid (e.g., cholesterol), or a PEG-modified lipid.
   75. The pharmaceutical composition, method, or kit of any one of embodiments 71-74, wherein the LNP comprises a second lipid.
   76. The pharmaceutical composition, method, or kit of embodiment 75, wherein the second lipid comprises a cationic lipid, a non-cationic (e.g., neutral, anionic, or zwitterionic) lipid, or an ionizable lipid.
   77. The pharmaceutical composition, method, or kit of any one of the preceding embodiments, wherein the composition (e.g., the pharmaceutical composition or the composition of (b)) or kit comprises a plurality of the DNA molecules.
   78. The pharmaceutical composition, method, or kit of any of embodiment 77, wherein:
(i) at least 50%, at least 60%, or at least 70% of the DNA molecules in the plurality have substantially the same length;
(ii) at least 50%, at least 60%, or at least 70% of the DNA molecules in the plurality have a length in a predetermined range; or
(iii) at least 50%, at least 60%, or at least 70% of the DNA molecules in the plurality have a length of between 100, 200, 300, 400, or 500 nucleotides of each other.
   79. The pharmaceutical composition, method, or kit of embodiment 77 or 78, wherein at least 50%, at least 60%, or at least 70% of the DNA molecules in the plurality have substantially the same sequence.
   80. The pharmaceutical composition, method, or kit of any one of the preceding embodiments, wherein the composition (e.g., the pharmaceutical composition or the composition of (a)) or kit comprises a plurality of the mRNA molecules.
   81. The pharmaceutical composition, method, or kit of embodiment 80, wherein:
(i) at least 50%, at least 60%, or at least 70% of the mRNA molecules in the plurality have substantially the same length;
(ii) at least 50%, at least 60%, or at least 70% of the mRNA molecules in the plurality have a length in a predetermined range; or
(iii) at least 50%, at least 60%, or at least 70% of the mRNA molecules in the plurality have a length of between 100, 200, 300, 400, or 500 nucleotides of each other.
   82. The pharmaceutical composition, method, or kit of embodiment 80 or 81, wherein at least 50%, at least 60%, or at least 70% of the mRNA molecules in the plurality have substantially the same sequence.
   83. The pharmaceutical composition, method, or kit of any one of the preceding embodiments, wherein the composition (e.g., the pharmaceutical composition or the composition of (a) and (b)) or kit induces an immune response in a subject to the first immunogen.
   84. The pharmaceutical composition, method, or kit of any one of the preceding embodiments, wherein the composition (e.g., the pharmaceutical composition or the composition of (a) and (b)) induces a humoral immune response in a subject to the first immunogen, e.g., 35 or 56 days after administration.
   85. The pharmaceutical composition, method, or kit of any one of the preceding embodiments, wherein the composition (e.g., the pharmaceutical composition or the composition of (a) and (b)) induces cellular immunity in a subject to the first immunogen, e.g., about 28, 35, 42, 56 or 70 days after administration.
   86. The pharmaceutical composition, method, or kit of any one of the preceding embodiments, which induces a humoral immune response in a subject to the first immunogen, wherein the humoral immune response is greater than a humoral immune response induced in a control subject that received (a) but not (b).
   87. The method of any of the preceding embodiments, comprising:
(i) administering to a subject a first dose comprising the composition that comprises the double stranded DNA molecule, and
(ii) administering to the subject a second dose comprising the composition that comprises the mRNA molecule, optionally wherein the second dose is administered 14 days to 28 days, e.g., about 21 days, after the first dose is administered.
   88. The method of any of embodiments 1-86, comprising:
(i) administering to a subject a first dose comprising the composition that comprises the mRNA molecule and the composition that comprises the double stranded DNA molecule, and
(ii) administering to the subject a second dose comprising the composition that comprises the mRNA molecule and the composition that comprises the double stranded DNA molecule, optionally wherein the second dose is administered 7 days to 21 days, e.g., about 14 days, after the first dose is administered.
   89. The method of any of the preceding embodiments, wherein the subject is a mammalian subject.
   90. The method of any of the preceding embodiments, wherein the subject is a human subject.
   91. The method of any of the preceding embodiments, wherein the subject is administered the composition via intramuscular administration.
   92. The method of any of the preceding embodiments, which results in a humoral response in the subject.
   93. The method of any of the preceding embodiments, which results in a cellular response in the subject.
   94. The method of any of the preceding embodiments, which results in a higher level of antigen-specific immune cell response in a subject, e.g., as measured using an ELIspot assay for IFN gamma levels.
   95. The method of any of embodiments 92-94, wherein the result is measured at 28, 35, 42, 56 or 70 days after the sole administration or the first administration.
   96. The composition or kit of any of the preceding embodiments, which results in a higher humoral neutralization response (e.g., a higher blood plasma 50% neutralizing titer (NT50) value) in a subject, as compared to a subject administered the mRNA molecule without the double stranded DNA molecule, as measured at 35 days after administration, e.g., as measured using a bead-based neutralization assay.
   97. A method of making the pharmaceutical composition or kit of any of the preceding embodiments, the method comprising: providing (a), providing (b), and admixing (a) with (b).
   98. The method of embodiment 97, which further comprises contacting the pharmaceutical composition or kit with one or both of a pharmaceutically acceptable excipient and a pharmaceutically acceptable diluent.
   99. The pharmaceutical composition, method, or kit of any of the preceding embodiments, wherein the first immunogen comprises a COVID protein, e.g., a COVID spike peptide.
   100. The pharmaceutical composition, method, or kit of any of the preceding embodiments, wherein the first immunogen comprises the amino acid sequence of SEQ ID NO: 1, or an amino acid sequence having at least 80%, at least 85%, at least 90%, at least 95%, at least 98%, or at least 99% identity thereto.
   101. The pharmaceutical composition, method, or kit of any of the preceding embodiments, wherein the second immunogen comprises a COVID protein, e.g., a COVID spike peptide.
   102. The pharmaceutical composition, method, or kit of any of the preceding embodiments, wherein the second immunogen comprises the amino acid sequence of SEQ ID NO: 1, or an amino acid sequence having at least 80%, at least 85%, at least 90%, at least 95%, at least 98%, or at least 99% identity thereto.
   103. The pharmaceutical composition or kit of any of the preceding embodiments for use in inducing an immune response in a subject.
   104. Use of the pharmaceutical composition or kit of any of the preceding embodiments in inducing an immune response in a subject.
   105. Use of the pharmaceutical composition or kit of any of the preceding embodiments in the manufacture of a medicament for inducing an immune response in a subject.
   106. The pharmaceutical composition, method, or kit of any of the preceding embodiments, wherein the dsDNA molecule is a circular.
   107. The pharmaceutical composition, method, or kit of any of the preceding embodiments, wherein the dsDNA molecule comprises a first strand and a second strand, and wherein the first strand comprises one or more chemically modified nucleobases, and the second strand is substantially free of (e.g., is free of) chemically modified nucleobases.
   108. The pharmaceutical composition, method, or kit of embodiment 107, wherein the first strand is a sense strand and the second strand is an antisense strand.
   109. The pharmaceutical composition, method, or kit of embodiment 107 or 108, wherein the chemically modified nucleobase comprises a uracil nucleobase.
   110. The pharmaceutical composition, method, or kit of any of embodiments 107-109, wherein at least 1%, at least 5%, at least 10%, at least 15%, at least 20%, at least 25%, at least 50%, at least 75%, at least 90%, at least 95%, at least 96%, at least 97%, at least 98%, or at least 99% of thymine or uracil positions in the first strand (e.g., sense strand) of the dsDNA molecule comprise the uracil nucleobase.
   111. The pharmaceutical composition, method, or kit of embodiment 109 or 110, wherein the uracil nucleobase is a canonical uracil nucleobase.

### Definitions

As used herein, the term “5′ untranslated region” (5′ UTR) refers to a region of an mRNA or pre-mRNA that is transcribed but not translated, and is 5′ of the coding region. Similarly, the term “3′ untranslated region” (3′ UTR) refers to a region of an mRNA or pre-mRNA that is transcribed but not translated, and is 3′ of the coding region.

As used herein, the term “antibody” refers to a molecule that specifically binds to, or is immunologically reactive with, a particular antigen and includes at least the variable domain of a heavy chain of an immunoglobulin. An antibody often also includes the variable domain of a light chain of an immunoglobulin. In some embodiments, the antibody is a full-length antibody, and in other embodiments, the antibody is a fragment of a full-length antibody. Antibodies include, but are not limited to, polyclonal, monoclonal, multispecific, human, humanized, primatized, or chimeric antibodies, heteroconjugate antibodies (e.g., bi-tri- and quad-specific antibodies, diabodies, triabodies, and tetrabodies), single-domain antibodies (sdAb), epitope-binding fragments, e.g., Fab, Fab′ and F(ab′) 2, Fd, Fvs, single-chain Fvs (scFv), rlgG, single-chain antibodies, disulfide-linked Fvs (sdFv), nanobody, fragments including either a VL or VH domain, fragments produced by an Fab expression library, and anti-idiotypic (anti-Id) antibodies. Antibodies described herein can be of any type (e.g., IgG, IgE, IgM, IgD, IgA, and IgY), class (e.g., IgG1, IgG2, IgG3, IgG4, IgAQ1 and IgA2) or subclass of immunoglobulin molecule.

As used herein, the term “antigen” refers to a polypeptide that binds an antigen binding site of an antibody or an antigen binding site of a TCR. In some embodiments, the antigen is an immunogen. The antigen may comprise an epitope or a plurality of epitopes.

As used herein, the term “bacterial antigen” refers to an antigen that comprises a bacterial amino acid sequence (e.g., a full-length wild-type bacterial protein or a portion of the full-length bacterial protein), or an amino acid sequence having sufficient sequence similarity to the bacterial amino acid sequence that it generates an immune response that recognizes the bacterial amino acid sequence. In some embodiment, the bacterial antigen has at least 90%, at least 95%, at least 98%, or at least 99% amino acid sequence identity to a wild-type bacterial amino acid sequence.

As used herein, the term “viral antigen” refers to an antigen that comprises a viral amino acid sequence (e.g., a full-length wild-type viral protein or a portion of the full-length viral protein), or an amino acid sequence having sufficient sequence similarity to the viral amino acid sequence that it generates an immune response that recognizes the viral amino acid sequence. In some embodiment, the viral antigen has at least 90%, at least 95%, at least 98%, or at least 99% amino acid sequence identity to a wild-type viral amino acid sequence.

As used herein, the term “antigen binding site” of an antibody or a TCR refers to the portion of the antibody or TCR that binds to an antigen. Typically, in an antibody, the antigen binding site comprises at least three complementarity determining regions (CDRs), and often six CDRs. Typically, in a TCR, the antigen binding site comprises three CDRs of the TCR α-chain and three CDRs of the TCR β-chain.

As used herein, the term “carrier” means a compound, composition, reagent, or molecule that facilitates or promotes the transport or delivery of a composition into a cell. For example, a carrier may be a partially or completely encapsulating agent.

As used herein, the term “chemically modified nucleotide,” as used herein with respect to DNAs, refers to a nucleotide comprising one or more structural differences relative to the canonical deoxyribonucleotides (i.e., G, T, C, and A). A chemically modified nucleotide may have (relative to a canonical nucleotide) a chemically modified nucleobase, a chemically modified sugar, a chemically modified phosphodiester linkage, or a combination thereof. No particular process of making is implied; for instance, a chemically modified nucleotide can be produced directly by chemical synthesis, or by covalently modifying a canonical nucleotide.

As used herein, the term “chemically modified cytosine nucleobase,” as used herein with respect to DNAs, refers to a chemically modified nucleobase wherein the nucleobase comprises a monocyclic 6-member ring in which carbon 4 is covalently bound to a nitrogen that is not one of the six members of the ring, wherein the nucleobase comprises one or more structural differences relative to canonical cytosine nucleobase. In some embodiments, the C-5 position of the nucleobase can have a substitution other than H. For example, the C-5 position of the nucleobase can have a substitution of —OH; -aldehyde; -carboxylic acid; -alkyl; —(CH₂)ₘOR₃, m=1-3 and R₃═H or a sugar molecule; orpropargylamino. No particular process of making is implied.

As used herein, the term “chemically modified uracil nucleobase,” as used herein with respect to DNAs, refers to a chemically modified nucleobase wherein the nucleobase comprises a monocyclic 6-member ring in which carbon 4 is covalently bound to an oxygen through a double bond, and wherein the nucleobase comprises one or more structural differences relative to canonical uracil and thymine nucleobases. In some embodiments, the C-5 position of the nucleobase can have a substitution other than H or a methyl group. For example, the C-5 position of the nucleobase can have a substitution of CH₂OH; -aminoallyl; or -propargylamino. No particular process of making is implied.

As used herein, the term “uracil nucleobase” encompasses both canonical uracil nucleobases and chemically modified uracil nucleobases.

As used herein, the term “chemically modified ribonucleotide,” as used herein with respect to RNAs, refers to a ribonucleotide comprising one or more structural differences relative to the canonical ribonucleotides (i.e., G, C, A, and U). A chemically modified ribonucleotide may have (relative to a canonical ribonucleotide) a chemically modified nucleobase, a chemically modified sugar, a chemically modified phosphodiester linkage, or a combination thereof. No particular process of making is implied; for instance, a chemically modified ribonucleotide can be produced directly by chemical synthesis, or by covalently modifying a canonical ribonucleotide.

As used herein the term “circular” in reference to a double-stranded DNA (dsDNA) molecule described herein, means a dsDNA molecule that lacks a free end. A circular dsDNA molecule may be covalently closed. The term circular does not imply that the DNA would appear as a perfect geometric circle under a microscope; for instance, a circular dsDNA molecule may be supercoiled. In some embodiments, the circular dsDNA molecule comprises a first strand that is circular and lacks a free end, and a second strand that is circular and lacks a free end, and the first strand and second strand hybridize with each other.

As used herein, the term “free end” in reference to a DNA molecule described herein, refers to an end of a DNA strand where the terminal nucleotide is covalently bound to exactly one other nucleotide.

As used herein, the term “closed end” refers to a portion of a DNA molecule positioned at one end of a double-stranded region, in which all nucleotides within the portion of the DNA molecule are covalently attached to adjacent nucleotides on either side. A closed end may, in some embodiments, include a loop comprising one or more nucleotides that are not hybridized to another nucleotide. In some embodiments (involving “no-loop” ends), the DNA end form is simply a covalent bond between the 5′ most nucleotide of the sense strand and the 3′ most nucleotide of the antisense strand, in the case of an upstream closed end, or the 3′ most nucleotide of the sense strand and the 5′ most nucleotide of the antisense strand, in the case of a downstream closed end. In some embodiments, a dsDNA molecule comprises a first closed end (e.g., upstream of a heterologous object sequence) and a second closed end (e.g., downstream of a heterologous object sequence).

As used herein, the term “immunogen” refers to a polypeptide that elicits an immune response. In some embodiments, the immune response comprises a humoral immune response and/or a cellular immune response. The immunogen may comprise an epitope or a plurality of epitopes.

As used herein, the term “open end” refers to a portion of a DNA molecule positioned at one end of a double-stranded region, in which at least one nucleotide (a “terminal nucleotide”) is covalently attached to exactly one other nucleotide. In some embodiments, the terminal nucleotide comprises a free 5′ phosphate. In some embodiments, the terminal nucleotide comprises a free 3′ OH. In some embodiments, in a double-stranded DNA molecule comprising a first DNA strand and a second DNA strand, the open end comprises a first terminal nucleotide on the first DNA strand and a second terminal nucleotide on the second DNA strand. In some embodiments, a DNA molecule comprises a first open end (e.g., upstream of an effector sequence) and a second open end (e.g., downstream of an effector sequence). In some embodiments, the open end comprises a blunt end, a sticky end, or a Y-adaptor.

As used herein, the term “DNA molecule” refers to any compound and/or substance that comprises at least two (e.g., at least 10, at least 20, at least 50, at least 100) covalently linked deoxyribonucleotides. In some embodiments, the DNA molecule is a single oligonucleotide chain, while in other embodiments, the DNA molecule comprises a plurality of oligonucleotide chains, while in yet other embodiments the DNA molecule is a portion of an oligonucleotide chain. In some embodiments, DNA molecule is a compound and/or substance that is or can be incorporated into an oligonucleotide chain via a phosphodiester linkage. In some embodiments, the DNA molecule comprises solely canonical nucleotides. In some embodiments, the DNA molecule comprises one or more chemically modified nucleotides. In some embodiments, at least 80%, at least 85%, at least 90%, at least 95%, at least 98%, at least 99%, or 100% of the sugars of the DNA molecule are deoxyribose sugars. In some embodiments, the DNA molecule was prepared by one or more of: isolation from a natural source, enzymatic synthesis by polymerization based on a complementary template (in vivo or in vitro), reproduction in a recombinant cell or system, and chemical synthesis.

As used herein, the term “DNA end form” refers to a structure comprising DNA that is situated at an end of a double-stranded DNA molecule. In some embodiments, the DNA end form comprises a closed end. In other embodiments, the DNA end form comprises an open end. In some embodiments, the DNA end form comprises a loop, a Y-adaptor, a blunt end, or a sticky end. The DNA end form may comprise one or both of a single stranded region and a double stranded region. The DNA end form may comprise canonical nucleotides, chemically modified nucleotides, or a combination thereof. In some embodiments, the DNA end form comprises between 3-100 nucleotides. In some embodiments, the double-stranded DNA molecule comprises a first DNA end form at a first end and a second DNA end form at a second end. In some embodiments, the first DNA end form and the second DNA end form of a DNA molecule are the same type. In some embodiments, the first DNA end form and the second DNA end form of a DNA molecule are different types.

As used herein, the term “effector sequence” of a DNA molecule refers to the part of a DNA molecule that exerts a function on a cell, either directly (wherein the effector sequence is a functional DNA sequence) or by encoding a functional RNA or protein. The encoded functional RNA or protein is referred to as the “effector”.

As used herein, the term “exonuclease-resistant”, when used to describe a DNA molecule, means that the DNA molecule, if it comprises closed ends, is resistant to the exonuclease assay as described in Example 10 or Example 11 of WO/2023/220729.

As used herein, the term “heterologous”, when used to describe a first element in reference to a second element means that the first element and second element do not exist in nature disposed as described. For example, a heterologous polypeptide, nucleic acid molecule, construct or sequence refers to (a) a polypeptide, nucleic acid molecule or portion of a polypeptide or nucleic acid molecule sequence that is not native to a cell in which it is expressed, (b) a polypeptide or nucleic acid molecule or portion of a polypeptide or nucleic acid molecule that has been altered or mutated relative to its native state, or (c) a polypeptide or nucleic acid molecule with an altered expression as compared to the native expression levels under similar conditions. For example, a heterologous regulatory sequence (e.g., promoter sequence or enhancer sequence) may be used to regulate expression of a gene or a nucleic acid molecule in a way that is different than the gene or a nucleic acid molecule is normally expressed in nature. In another example, a heterologous domain of a polypeptide or nucleic acid sequence (e.g., a DNA binding domain of a polypeptide or nucleic acid encoding a DNA binding domain of a polypeptide) may be disposed relative to other domains or may be a different sequence or from a different source, relative to other domains or portions of a polypeptide or its encoding nucleic acid. In certain embodiments, a heterologous nucleic acid molecule may exist in a native host cell genome, but may have an altered expression level or have a different sequence or both. In other embodiments, heterologous nucleic acid molecules may not be endogenous to a host cell or host genome but instead may have been introduced into a host cell by transformation (e.g., transfection, electroporation), wherein the added molecule may integrate into the host genome or can exist as extra-chromosomal genetic material either transiently (e.g., mRNA) or semi-stably for more than one generation (e.g., episomal viral vector, plasmid or other self-replicating vector).

As used herein, the term “heterologous functional sequence” refers to a nucleic acid sequence that is heterologous to an adjacent (e.g., directly adjacent) nucleic acid sequence and has one or more biological function.

As used herein, the terms “increasing” and “decreasing” refer to modulating resulting in, respectively, greater or lesser amounts, of function, expression, or activity of a metric relative to a reference. For example, subsequent to administration of a composition described herein in a method described herein, the amount of the metric described herein (e.g., the level of gene expression, or a marker of innate immunity) may be increased or decreased in a subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 98% or more relative to the amount of the marker prior to administration, or relative to administration of a control composition. Generally, the metric is measured subsequent to administration at a time that the administration has had the recited effect, e.g., one day, one week, one month, 3 months, or 6 months, after a treatment regimen has begun.

As used herein, the term “linear” in reference to a double-stranded DNA molecule described herein, means a nucleic acid comprising two DNA strands or portions of strands which hybridize with each other (thereby forming a double stranded region), wherein the structure comprises two ends. An end may be a closed end or an open end. The two strands that hybridize with each other may be partially or completely complementary. In some embodiments, a linear double-stranded DNA molecule consists of a single strand of DNA that, under denaturing conditions, does not form any double stranded regions and does not have any free ends, and wherein under physiological conditions a first portion of the strand hybridizes to a second portion of the strand (thereby forming a double stranded region), and the linear double-stranded DNA molecule comprises a first closed end comprising a first loop and a second closed end comprising a second loop.

As used herein, when two entities are “linked”, the two entities are physically connected by means of one or more covalent or noncovalent bond. In some embodiments, the two entities are directly linked, i.e., an atom of the first entity forms a covalent or noncovalent bond with an atom of the second entity. In some embodiments, the two entities are indirectly linked through a third entity; for example A is linked to C by virtue of A being directly linked to B and B being directly linked to C.

As used herein, the terms “linker” refers to a moiety that connects two parts of a compound, e.g., covalently attaches two parts of a compound.

As used herein, the term “loop” refers to a nucleic acid sequence that is single stranded. A loop is connected at both ends by a double stranded region referred to as a “stem”, to form a “stem-loop”.

As used herein, the term “maintenance sequence” is a DNA sequence or motif that enables or facilitates retention of a DNA molecule in the nucleus through cell division. A maintenance sequence typically enables replication and/or transcription of DNA in the nucleus by interacting with proteins that facilitate chromatin looping. An example of a maintenance sequence is a scaffold/matrix attached region (S/MAR element).

As used herein, a “pharmaceutical composition” or “pharmaceutical preparation” is a composition or preparation which is indicated for animal, e.g., human or veterinary pharmaceutical use, for example, non-human animal or human prophylactic or therapeutic use. A pharmaceutical preparation comprises an active agent having a biological effect on a cell or tissue of a subject, e.g., having pharmacological activity or an effect in the mitigation, treatment, or prevention of disease, in combination with a pharmaceutically acceptable excipient or diluent.

A pharmaceutical composition also means a finished dosage form or formulation of a prophylactic or therapeutic composition.

As used herein, the terms “peptide,” “polypeptide,” and “protein” are used interchangeably and refer to a compound comprising amino acid residues covalently linked by peptide bonds, or by means other than peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types. In some embodiments, a polypeptide comprises a non-canonical amino acid residue.

As used herein, the term “polyadenylation site” refers to a segment of an mRNA or pre-mRNA that is cleaved and polyadenylated. A polyadenylation site typically comprises the sequence CA.

As used herein, the term “polyadenylation signal” refers to a segment of an mRNA or pre-mRNA that recruits polyadenylation machinery. A polyadenylation signal typically comprises the sequence AAUAAA.

As used herein, a “sense strand” of a DNA molecule is a strand which has the same sequence as an mRNA or pre-mRNA which encodes for a functional RNA or protein, and does not serve as a template for transcription. An “antisense strand” of a DNA molecule is a strand that has a sequence complementary to an mRNA or pre-mRNA which encodes for a functional RNA or protein and/or can serve as a template for transcription.

As used herein, the term “double-stranded DNA molecule” or dsDNA molecule means a DNA composition comprising two complementary chains of deoxyribonucleotides that base pair to each other. The two complementary strands may have perfect complementarity or may have one or more mismatches, e.g., forming bulges. Either of the two strands may, in some embodiments, have paired regions of self-complementarity that form intramolecular/intrastrand double stranded motifs in a folded configuration, for example, may form hairpin loops, junctions, bulges or internal loops. In some embodiments, the dsDNA molecule comprises one or two closed ends. In some embodiments, the dsDNA molecule is circular or linear. In some embodiments (e.g., in a dsDNA molecule with closed ends) the two complementary chains of deoxyribonucleotides are covalently linked.

As used herein, the term “therapeutic double stranded construct” (“TDSC”) refers to a linear construct comprising DNA, wherein the construct is at least partially double stranded. A TDSC does not comprise a plasmid backbone sequence (e.g., does not comprise a bacterial origin of replication). A TDSC does not comprise a viral capsid or a viral envelope. In some embodiments, the TDSC comprises a closed end or an open end (e.g., a blunt end or a sticky end). In some embodiments, the TDSC is suitable for administration to a human subject.

As used herein, the term “terminal nucleotide” refers to a nucleotide that is covalently attached to exactly one other nucleotide. In some embodiments, the terminal nucleotide comprises a free 5′ phosphate. In some embodiments, the terminal nucleotide comprises a free 3′ OH.

As used herein, “treatment” and “treating” refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “Y-adaptor” refers to a nucleic acid structure comprising a first nucleic acid region and a second nucleic acid region which are complementary (e.g., perfectly complementary) to each other; the first and second regions may hybridize to form a double stranded region. The first nucleic acid region is covalently linked to a third nucleic acid region, and the second nucleic acid region is covalently linked to a fourth nucleic acid region, and the third and fourth nucleic acid regions are not substantially complementary to each other; the third and fourth regions may be single stranded. The first nucleic acid region is 3′ of the third nucleic acid region and the second nucleic acid region is 5′ of the fourth nucleic acid region. As a result, the third and fourth regions may be situated on the same side of the double stranded regions. The Y-adaptor may be part of a double-stranded DNA molecule.

### BRIEF DESCRIPTION OF THE DRAWINGS

FIG. 1 shows the blood plasma 50% neutralizing titer (NT50) following intramuscular (I.M.) vaccine administration in mice. Blood plasma was collected 35 days after the first dose of the vaccine.

FIGS. 2A and 2B show humoral and cellular response following vaccine administration in mice. FIG. 2A shows blood plasma 50% neutralizing titer following I.M. vaccine administration in mice. Blood plasma was collected at either day 35 or day 56 following the first dose of the vaccine. FIG. 2B shows the percentage of CD4 or CD8 T cells that specifically recognized Covid Spike isolated from dissociated spleen. Tissue was harvested at day 35 following the first dose. The y-axis represents the percentage of Covid Spike peptide-specific central memory CD4+ T cells (left graph) or Covid Spike peptide-specific central memory CD8+ T cells (right graph) of the CD4+ T cells or CD8+ T cells (respectively) isolated from the spleen.

FIG. 3 shows blood plasma 50% neutralizing titer following I.M. vaccine administration in mice. Blood plasma was collected 35 days after the first dose of vaccine administration. Mice were administered mRNA encoding the antigen (Ag) and DNA encoding the antigen (Ag) or control DNA that does not encode the antigen (Ctrl).

FIG. 4 show antigen-specific cellular responses against Covid Spike, as assayed via IFN gamma (IFNg) ELISpot assay, of peripheral blood mononuclear cells (PBMCs) isolated from cynomolgus macaque non-human primates. The animals were administered a first dose of an mRNA vaccine on study day 1 and a second dose of an mRNA vaccine on study day 22 (indicated with “mRNA mRNA”), or a dose of a DNA vaccine on day 1 and a dose of an mRNA vaccine on day 22 (indicated with “DNA mRNA”). The mRNA and DNA vaccines encoded a Covid spike peptide. FIG. 4 shows quantification of IFNg ELIspot spot forming units (SFU) per 1e6 cells. Data is shown for PBMCs collected pre-dose or day 29, 36, 43, 57 and 71 following initial dose.

### DETAILED DESCRIPTION

This disclosure provides a composition (e.g., a pharmaceutical composition) comprising an mRNA molecule encoding a first immunogen, and a DNA molecule encoding: (i) the first immunogen, (ii) a second immunogen having at least 90%, at least 95%, at least 98%, or at least 99% amino acid sequence identity to the first immunogen, or (iii) a second immunogen that is recognized by an antigen binding site of an antibody or T cell receptor (TCR), wherein the antigen binding site also recognizes the first immunogen. In some embodiments, the composition provided herein elecits, or is capable of eleciting, an immune response, e.g., in a subject, targeting the first immunogen. In some embodiments, the immune response induced by the composition is greater than an immune response induced, e.g., in the subject, by the mRNA molecule or the DNA molecule alone.

In some aspects, the composition (e.g., pharmaceutical composition) disclosed herein is a vaccine. In some aspects, the vaccine may be used for preventing and/or treating infection in a subject. In certain aspects, the vaccine is used in the treatment and/or prevention of a disease. In embodiments, the vaccine triggers a T-cell specific response when administered to a subject. In embodiments, the vaccine triggers a B-cell specific response when administered to a subject. This disclosure also relates to compositions and methods for providing an effector, e.g., a therapeutic effector, to a cell, tissue or subject, e.g., in vivo or in vitro. The effector may be a polypeptide, e.g., an immunogen.

In some aspects, the present disclosure provides a pharmaceutical composition comprising (a) an mRNA molecule encoding an immunogen; and (b) a circular double stranded DNA (dsDNA) molecule encoding the immunogen and comprising a first strand and a second strand, wherein the first strand comprises one or more chemically modified nucleobases, and the second strand is free of chemically modified nucleobases.

### Elements of DNA Molecules

The DNA molecules described herein can contain elements sufficient to deliver an effector sequence to a target cell, tissue or subject. In some embodiments, the DNA molecule drives expression of an effector, e.g., the DNA molecule comprises a promoter sequence and a sequence encoding a polypeptide, e.g., a therapeutic polypeptide. In some embodiments, the DNA molecules described herein further contain a maintenance sequence.

In some embodiments, the DNA molecule comprises a double-stranded DNA molecule. In some embodiments, the DNA molecule comprises a circular double-stranded DNA molecule. In some embodiments, the circular double-stranded DNA molecule comprises a plasmid or a minicircle. In some embodiments, the DNA molecule comprises a linear double-stranded DNA molecule (e.g., TDSC).

In some embodiments, a DNA molecule disclosed herein is at least about 10 nucleotides, at least about 15 nucleotides, at least about 20 nucleotides, at least about 25 nucleotides, at least about 30 nucleotides, at least about 40 nucleotides, at least about 50 nucleotides, at least about 60 nucleotides, at least about 70 nucleotides, at least about 75 nucleotides, at least about 80 nucleotides, at least about 90 nucleotides, at least about 100 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 2000 nucleotides, at least about 3000 nucleotides, at least about 4000 nucleotides, at least about 5000 nucleotides, at least about 6000 nucleotides, at least about 7000 nucleotides, at least about 8000 nucleotides, at least about 9000 nucleotides, at least about 10,000 nucleotides, at least about 11,000, or at least about 12,000 nucleotides in length. In some embodiments, a DNA molecule disclosed herein is less than 50, less than 60, less than 70, less than 80, less than 90, less than 100, less than 200, less than 300, less than 400, less than 500, less than 600, less than 700, less than 800, less than 900, less than 1000, less than 2000, less than 3000, less than 4000, less than 5000, less than 6000, less than 7000, less than 8000, less than 9000, less than 10,000, less than 11,000, or less than 12,000 nucleotides in length. In some embodiments, a DNA molecule disclosed herein is between 10-15, 15-20, 20-25, 25-30, 30-35, 35-40, 40-45, 45-50, 50-55, 55-60, 60-70, 70-80, 80-90, 90-100, 20-30, 30-40, 40-50, 50-75, 75-100, 100-200, 200-300, 300-400, 400-500, 300-500, 500-600, 600-700, 700-800, 800-900, 900-1000, 500-1000, 1000-2000, 2000-3000, 3000-4000, 4000-5000, 5000-6000, 6000-7000, 7000-8000, 8000-9000, 9000-10,000, 10,000-11,000, or 11,000-12,000 nucleotides in length. In some embodiments, the size of a DNA molecule disclosed herein is a length sufficient to encode useful polypeptides or RNAs.

In some embodiments, a DNA molecule described herein is resistant to endonuclease digestion and/or resistant to immune sensor recognition. In some embodiments, at least 80%, at least 85%, at least 90%, at least 95%, at least 98%, at least 99%, or 100% of the sugars of a DNA molecule described herein are deoxyribose sugars.

In some embodiments, a DNA molecule described herein can be replicated (e.g., by a DNA polymerase native to a cell comprising the DNA molecule). In some embodiments, a DNA molecule described herein cannot be replicated. In some embodiments, a DNA molecule or a portion thereof can be integrated into the genome. In some embodiments, a DNA molecule or a portion thereof cannot be integrated into the genome.

In some embodiments, a double-stranded DNA molecule comprises an exonuclease-resistant DNA end form (e.g., as described herein). In some embodiments, the DNA end form is at least 2, at least 3, at least 4, at least 5, at least 10, at least 15, at least 20, at least 25, at least 30, at least 40, at least 50, at least 60, at least 70, at least 80, at least 90, or at least 100 nucleotides in length. In some embodiments, the DNA end form is less than 10, less than 15, less than 20, less than 25, less than 30, less than 40, less than 50, less than 60, less than 70, less than 80, less than 90, or less than 100 nucleotides in length. In some embodiments, the DNA end form is 2-5, 5-10, 10-15, 15-20, 20-25, 25-30, 30-35, 35-40, 40-45, 45-50, 50-55, 55-60, 60-70, 70-80, 80-90, or 90-100 nucleotides in length. It is understood that when the length of a linear closed-ended dsDNA molecule is discussed herein, the length refers to the number of nucleotides starting with and including the upstream end, through the downstream end. For example, a no-loop dsDNA molecule having 100 base pairs would have a length of 100 nucleotides.

In some embodiments, a double-stranded DNA molecule comprises an effector sequence encoding an effector (e.g., a polypeptide, e.g., as described herein), e.g., positioned between two exonuclease-resistant DNA end forms.

A double-stranded DNA molecule described herein may have less than a threshold level of single stranded structures. In one embodiment, the double-stranded DNA molecule does not comprise more than 20, 18, 16, 14, 12, 10, 8, 7, 5, 4, 3, 2, or 1 single stranded region longer than 100, 80, 70, 60, 50, 40, 30, 20 or 10 bases, e.g., does not comprise single stranded regions longer than 100, 80, 70, 60, 50, 40, 30, 20 or 10 bases.

In some embodiments, a double-stranded DNA molecule comprises a sense strand and an antisense strand.

In some embodiments, a DNA molecule comprises (a) an upstream end form (e.g., upstream exonuclease-resistant DNA end form); (b) double-stranded DNA; and (c) a downstream end form (e.g., exonuclease-resistant DNA end form). In some embodiments, the upstream exonuclease-resistant DNA end form has a loop size of less than about 28 or 56 nucleotides in length or greater than about 28 or 56 nucleotides in length. In some embodiments, the downstream exonuclease-resistant DNA end form has a loop size of less than about 28 or 56 nucleotides in length or greater than about 28 or 56 nucleotides in length. In some embodiments, every nucleotide in the DNA molecule binds another nucleotide in the DNA molecule.

In some embodiments, the upstream DNA end form and the downstream DNA end form have the same nucleotide sequence. In some embodiments, the upstream DNA end form and the downstream DNA end form have different nucleotide sequences. In some embodiments, the upstream exonuclease-resistant DNA end form and the downstream exonuclease-resistant DNA end form have the same structure. In some embodiments, the upstream exonuclease-resistant DNA end form and the downstream exonuclease-resistant DNA end form have different structures.

In some embodiments, a double-stranded DNA molecule described herein is linear and can be circularized. In some embodiments, a double-stranded DNA molecule described herein is linear and cannot be circularized. In some embodiments, a double-stranded DNA molecule described herein can be concatemerized. In some embodiments, a double-stranded DNA molecule described herein cannot be concatemerized.

### Exonuclease-Resistant DNA End Forms

In some embodiments, a double-stranded DNA molecule described herein comprise a DNA end form at each end of the double-stranded DNA molecule. The DNA end forms described herein can, in some instances, comprise a closed end, wherein every nucleotide of the DNA end form is covalently attached to two other nucleotides of the DNA end form. In other instances, the DNA end forms described herein comprise an open end comprising at least one nucleotide that is only covalently attached to one other nucleotide of the DNA end form. The DNA end forms are generally exonuclease resistant.

In some embodiments, a DNA molecule described herein comprises an upstream DNA end form which is a closed end; (b) a double stranded region; and (c) a downstream DNA end form which is a closed end.

Exemplary exonuclease-resistant DNA end forms, the production of exonuclease-resistant DNA end forms, and assessment of exonuclease resistance can be found, for example, in WO/2023/220729, incorporated herein by reference in its entirety.

### Loops

In some embodiments, a DNA end form comprises a loop.

The DNA end form comprising a loop may be produced, e.g., by ligating an end adaptor which is a hairpin to a dsDNA molecule. A hairpin generally comprises a single-stranded loop region covalently attached at both the 5′ and 3′ ends to a double-stranded stalk region. In certain embodiments, the single-stranded loop region comprises one or more nucleotides (e.g., 1-2, 2-5, 5-10, 10-15, 15-20, 20-25, 25-30, 30-35, or 35-40 nucleotides) that are not hybridized to another nucleotide. In embodiments, the loop is comprised in a DNA molecule having a doggybone conformation.

### Y-Adaptors

In some embodiments, an exonuclease-resistant DNA end form as described herein comprises a Y-adaptor. As described herein, a Y-adaptor generally comprises a pair of single-stranded DNA regions, each attached at one end to a strand of a double-stranded DNA region, thereby forming a “Y” shape (wherein the base of the “Y” represents the double-stranded DNA region, and each of the upper prongs of the “Y” represents the two single-stranded DNA region).

### No Loop Closed DNA End Forms

In some embodiments, a double-stranded DNA molecule as described herein comprises an exonuclease-resistant DNA end form that is covalently closed but does not include a single stranded loop. For example, in certain embodiments, every nucleotide of a covalently-closed DNA molecule is complementary to another nucleotide. Accordingly, the DNA end form may be a bond between the endmost nucleotide of the sense strand and the nucleotide of the antisense strand which base pairs with the nucleotide of the sense strand.

### Open DNA End Forms

In some embodiments, a double-stranded DNA molecule as described herein comprises an exonuclease-resistant DNA end form that is not covalently closed. In certain embodiments, the DNA end form comprises a blunt end (e.g., a blunt end comprising one or more chemical modifications as described herein) or a sticky end (e.g., a sticky end comprising one or more chemical modifications as described herein).

### Inverted Terminal Repeats (ITRs)

In some embodiments, a double-stranded DNA molecule as described herein comprises an exonuclease-resistant DNA end form comprising an inverted terminal repeat (ITR). In some embodiments, the ITR is an ITR from a virus, e.g., an adenovirus or an adeno-associated virus (AAV). In some embodiments, the ITR comprises a nucleic acid sequence having at least 75%, at least 80%, at least 85%, at least 90%, at least 95%, at least 96%, at least 97%, at least 98%, at least 99%, or 100% sequence identity to an ITR sequence from a virus, e.g., an adenovirus or an adeno-associated virus (AAV). In certain embodiments, the ITR comprises an origin of replication (e.g., a viral origin of replication). In embodiments, a double-stranded DNA molecule as described herein comprises an exonuclease-resistant DNA end form comprising an ITR (e.g., as described herein) at each end. In some embodiments, a double-stranded DNA molecule does not comprise an ITR.

### Sequence Elements of DNA Molecules

In some embodiments, a DNA molecule described herein comprises a promoter sequence. In some embodiments, a DNA molecule described herein comprises an effector sequence (e.g., a therapeutic effector sequence) operably linked to the promoter sequence. In some embodiments, a DNA molecule described herein comprises a heterologous functional sequence. In some embodiments, a DNA molecule described herein comprises a maintenance sequence. In some embodiments, a DNA molecule described herein comprises an origin of replication. In some embodiments, the DNA molecule comprises one, two, three, four, or all of a promoter sequence, an effector sequence, a heterologous functional sequence, a maintenance sequence, or an origin of replication. In some embodiments, the DNA molecule comprises a promoter sequence, an effector sequence, and a heterologous functional sequence. In some embodiments, the DNA molecule comprises a promoter sequence, an effector sequence, and a maintenance sequence. In some embodiments, the DNA molecule comprises a promoter sequence, an effector sequence, and an origin of replication. In some embodiments, the DNA molecule comprises a promoter sequence, an effector sequence, a heterologous functional sequence, and a maintenance sequence. In some embodiments, the DNA molecule comprises a promoter sequence, an effector sequence, a heterologous functional sequence, and an origin of replication. In some embodiments, the DNA molecule comprises a promoter sequence, an effector sequence, a maintenance sequence, and an origin of replication. In some embodiments, the DNA molecule comprises a promoter sequence, an effector sequence, a heterologous functional sequence, a maintenance sequence, and an origin of replication.

In some embodiments, a DNA molecule described herein comprises an effector sequence that encodes an effector. In some embodiments, the effector sequence encodes a polypeptide (e.g., a protein). In some embodiments, the effector sequence is heterologous to a target cell.

### Promoter Sequences and Other Regulatory Sequences

The DNA molecule described herein may contain a promoter sequence (a DNA sequence at which RNA polymerase and transcription factors bind to, directly or indirectly, to initiate transcription) operably linked to an effector sequence. A promoter sequence may be found in nature operably linked to the effector sequence, or may be heterologous to the effector sequence. A promoter sequence described herein may be native to the target cell or tissue, or heterologous to the target cell or tissue. A promoter sequence may be constitutive, inducible and/or tissue-specific.

Examples of constitutive promoter sequences include sequences of the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985), the dihydrofolate reductase promoter, the beta-actin promoter, the phosphoglycerol kinase (PGK) promoter, and the EF1alpha promoter.

Inducible promoter sequences allow regulation of expression and can be regulated by exogenously supplied compounds, environmental factors such as temperature, or the presence of a specific physiological state, e.g., acute phase, a particular differentiation state of the cell, or in replicating cells only. Inducible promoter sequences and inducible systems are available from a variety of sources. Examples of inducible promoter sequences regulated by exogenously supplied promoters include sequences of the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al, Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)).

In some embodiments, the native promoter sequence for the sequence encoding the effector can be used.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoter sequences, enhancer sequences, etc.) are known in the art. Exemplary tissue-specific promoter sequences include, but are not limited to, sequences of: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a alpha-myosin heavy chain (a-MHC) promoter, or a cardiac Troponin T (cTnT) promoter. Other exemplary promoter sequences include sequences of the Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor alpha-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be known to the skilled artisan.

Examples of tissue/cell specific promoter sequences are listed in Table 1:

TABLE 1
Tissue or cell specific promoter sequences

| | | Accession Number; Human |
|---|---|---|
| Tissue/Cell | Promoter | Genome Coordinate (hg38) |
| Skeletal | ACTA1 | NM_001100; chr1: 229,439,090- |
| muscle | | 229,432,090 |
| Melanoma | TYR | NM_000372; chr11: 89,300,750- |
| | | 89,293,750 |
| Hepatoma | a- | NM_001354717; chr4: 73,461,175- |
| | fetoprotein | 73,454,175 |
| Mammary | Mucin 1 | NM_001371720; |
| carcinoma | | chr1: 155,197,900-155,190,900 |
| Prostate | KLK3 | NM_001648; chr19: 50,865,760- |
| Cancer | | 50,858,760 |
| Neuronal | ENO2 | NM_001975; chr12: 6,928,700- |
| cells | | 6,921,700 |
| Response to | HIF- | NM_001530; chr14: 61,753,200- |
| Hypoxia | 1alpha | 61,746,200 |
| Retinoblastoma | E2F1 | NM_005225; chr20: 33,691,380- |
| | | 33,684,380 |
| Ionizing | EGR-1 | NM_001964; chr5: 138,474,303- |
| radiation | | 138,467,303 |
| Oncogene | ErbB2 | NM_004448; chr17: 39,735,530- |
| | | 39,728,530 |
| Endothelial | vWF | NM_000552; chr12: 6,129,670- |
| cells | | 6,122,670 |
| Endothelial | FLT-1 | NM_002019; chr13: 28,500,100- |
| cells | | 28,493,100 |
| Endothelial | ICAM-2 | NM_001099786; |
| cells | | chr17: 64,025,630-64,018,630 |
| Retinal | VMD2 | NM_004183; chr11: 61,972,630- |
| pigment | | 61,965,630 |
| epithelium | | |
| Rod cells | RHO | NM_000539; chr3: 129,540,350- |
| | | 129,533,350 |
| Cone cells | Red/green | NM_020061; chrX: 154,164,030- |
| | opsin | 154,157,030 |
| | (OPN1LW) | |
| Ganglion | Thymocyte | NM_006288; chr11: 119,428,150- |
| cells | antigen | 119,421,150 |
| | (Thy1) | |
| T cells | TIM3 | NM_032782; chr5: 157,114,050- |
| | | 157,107,050 |
| T cells | FOXP3 | NM_014009; chrX: 49,269,700- |
| | | 49,262,700 |
| PBMCs | Vβ6.7 | ENST00000390373.2; |
| | | chr7: 142,493,295-142,486,295 |
| Cell cycle | Cdk1 | NM_001786; chr10: 60,799,850- |
| | | 60,792,850 |

The DNA molecules described herein may also include other native or heterologous expression control elements, such as enhancer elements, a sequence encoding a polyadenylation site, or Kozak consensus sequences. In some embodiments, a DNA molecule described herein comprises a sequence encoding a polyadenylation site. In some embodiments, a DNA molecule described herein comprises a sequence encoding a polyadenylation signal.

### Effector Sequence

The effector sequence of a DNA molecule described herein may be, e.g., a DNA sequence encoding a therapeutic peptide, polypeptide or protein, such as an immunogen.

In some embodiments, a polypeptide effector encoded by DNA molecule described herein is an immunogen. For example, in some embodiments, the immunogen is a viral antigen (e.g., a coronavirus antigen or a lentivirus antigen). In some embodiments, the viral antigen is a COVID-19 antigen, a hepatitis antigen (e.g., a hepatitis B antigen or a hepatitis A antigen), an influenza antigen, an HPV antigen, a chickenpox antigen, a measles antigen, a mumps antigen, a rubella antigen, or a combination thereof. In some embodiments, the immunogen is a bacterial antigen. In some embodiments, the bacterial antigen is a diphtheria antigen, a tetanus antigen, a pertussis antigen, a meningitidis antigen, a tuberculosis antigen, a cholerae antigen, or a combination thereof. In some embodiments, the immunogen is a fungal antigen. In some embodiments, the antigen is a tumor antigen.

In some embodiments, the immunogen is a peptide. In some embodiments, the immunogen polypeptide has a length of 10-20, 20-50, 50-100, 100-500, 500-1000, 1000-2000, or 2000-5000 amino acids.

In some embodiments, the immunogen is not a pneumococcal polypeptide antigen, a Bacilli antigen, a Lactobacillus antigen, a Streptococcaceae antigen, a COVID antigen, a coronavirus antigen, an HIV antigen, or a lentivirus antigen.

In some embodiments, a DNA molecule described herein may include one or a plurality of sequences encoding a polypeptide, e.g., 2, 3, 4, 5, 6, or more sequences encoding a polypeptide. Each of the plurality may encode the same or different protein. For example, a sequence described herein may include multiple sequences encoding multiple proteins, e.g., a plurality of proteins in a biological pathway.

In some embodiments, a DNA molecule described herein may include a plurality of sequences encoding a polypeptide, e.g., 2, 3, 4, 5, 6, or more sequences encoding a polypeptide, separated by a self-cleaving peptide, e.g., P2A, T2A, E2A or F2A. Self-cleaving peptides are typically 18-22 amino acids long, and can induce ribosomal skipping during protein translation so that two polypeptides can be encoded in the same transcript. Each of the polypeptides may encode the same or different protein. In one embodiment, a DNA molecule described herein may include a promoter sequence followed by a sequence encoding a first polypeptide of interest, a sequence encoding a 2A self-cleaving peptide, a sequence encoding a second polypeptide of interest, and a sequence encoding polyA site. In another embodiment, a DNA molecule described herein may include a promoter sequence followed by a sequence encoding the first polypeptide of interest, a first 2A self-cleaving peptide, a second polypeptide of interest, a sequence encoding a second 2A self-cleaving peptide, a sequence encoding a third polypeptide of interest, and a sequence encoding polyA site.

In some embodiments, the effector sequence that encodes a polypeptide or protein is codon optimized, e.g., codon optimized for expression in a mammal, e.g., a human. In general, codon optimization means modifying a nucleic acid sequence for enhanced expression in the host cells of interest by replacing at least one codon (e.g., one or more, e.g., 1, 2, 3, 4, 5, 10, 15, 20, 25, 50, or more codons; e.g., at least 1%, at least 5%, at least 10%, at least 20%, at least 25%, at least 50%, at least 60%, at least 70%, at least 80%, at least 90% or 100%) of the native sequence with codons that are more frequently or most frequently used in the genes of that host cell while maintaining the native amino acid sequence. Codon usage tables are available, for example, at the “Codon Usage Database” available at www.kazusa.or.jp/codon/. These tables can be adapted in a number of ways, see, e.g., Nakamura et al., 2000, Nucl. Acids Res. 28:292. Computer algorithms for codon optimizing a particular sequence for expression in a particular host cell are also available, such as Gene Forge.

In some embodiments, an immunogen comprises a Covid spike peptide. An exemplary amino acid sequence of a Covid Spike peptide is provided below:

(SEQ ID NO: 1)
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDPPEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT

In some embodiments, the COVID spike peptide comprises the amino acid sequence of SEQ ID NO: 1, or an amino acid sequence having at least 85%, at least 90%, at least 95%, at least 96%, at least 97%, at least 98%, or at least 99% identity thereto.

### Maintenance Sequence

A DNA molecule disclosed herein may include a maintenance sequence that supports or enables sustained gene expression through successive rounds of cell division and/or progenitor differentiation in a host cell for the DNA molecule described herein. In embodiments, a maintenance sequence is a nuclear scaffold/matrix attachment region (S/MAR). S/MAR elements are diverse, AT-rich sequences ranging from 60-500 bp that are conserved across species, thought to anchor chromatin to nuclear matrix proteins during interphase (Bode et al. 2003. Chromosome Res 11, 435-445). An S/MAR can be incorporated into a DNA molecule described herein to facilitate long-term transgene expression and extra-chromosomal maintenance. In one embodiment, the maintenance sequence is human interferon-beta MAR (5′tataattcactggaatttttttgtgtgtatggtatgacatatgggttcccttttattttttacatataaatatatttccctgtttttctaaaaaagaaaa agatcatcattttcccattgtaaaatgccatatttttttcataggtcacttacata-3′ (SEQ ID NO: 2)), or a functional sequence having at least 80%, at least 90%, at least 95%, or at least 98% identity thereto. In embodiments, S/MARs useful in the constructs described herein can be found by searching the MARome at bioinfo.net.in/MARome, described also by Narwade et al. 2019. Nucleic Acids Research. Volume 47, Issue 14:7247-7261.

In embodiments, a DNA molecule described herein is capable of replicating in a mammalian cell, e.g., human cell. In some embodiments, a DNA molecule described herein is maintained in a host cell, tissue or subject through at least one cell division. For example, a DNA molecule described herein is maintained in a host cell, tissue or subject through at least 2, at least 3, at least 4, at least 5, at least 6, at least 7, at least 8, at least 10, at least 15, at least 20, at least 40, at least 50 or more cell divisions. In vitro, cell division may be tracked by flow cytometry or microscopy. In vivo, cell division may be tracked by intravital microscopy.

### Other Elements

A DNA molecule disclosed herein may also include other control elements operably linked to the effector sequence, e.g., the sequence encoding an effector, in a manner which permits its transport, localization, transcription, translation and/or expression in a target cell, or which promotes its degradation or repression of expression in a non-target cell. As used herein, “operably linked” sequences include both expression control sequences that are contiguous with the sequence encoding the effector and expression control sequences that act in trans or at a distance to control the sequence encoding the effector. The precise nature of regulatory sequences needed for gene expression in host cells may vary between species, tissues or cell types, but in general may include, as necessary, 5′ non-transcribed and 5′ non-translated sequences involved with the initiation of transcription and translation respectively, such as a TATA box, capping sequence, CAAT sequence, enhancer elements and the like. Regulatory sequences may also include enhancer sequences or upstream activator sequences as desired. The constructs described herein may optionally include 5′ leader or signal sequences. In some embodiments, a DNA molecule described herein may comprise a sequence encoding a 5′ untranslated region (5′ UTR) and/or a sequence encoding a 3′ untranslated region (3′ UTR). In some embodiments, the DNA molecule encodes an intron sequence. In some embodiments, the DNA molecule comprises a sequence encoding a polyadenylation site. In some embodiments, the DNA molecule comprises an enhancer sequence.

The DNA molecule may comprise a non-coding region. In some embodiments, the non-coding region is completely free of predicted ORFs. In some embodiments, the non-coding region does not encode a protein sequence. In some embodiments, the non-coding region is not translated or is not translated at a substantial level.

CpG Depletion in dsDNA Molecules

A double-stranded DNA molecule described herein may comprise a sequence that is CpG-depleted. A CpG of a dsDNA molecule refers to a dinucleotide region of the DNA molecule where a cytosine nucleotide is directly followed by a guanine nucleotide in the linear sequence of bases along the 5′ to 3′ direction. The “P” represents the phosphate group between the C and G. Without wishing to be bound by theory, the depletion of CpG's from a dsDNA molecule is thought to lead to a weakened immune response against the dsDNA molecule in a subject administered the CpG-depleted dsDNA. For instance, CpG depletion may lead to lower activation of IL-6 and/or IP-10. No particular method of making is implied in the term “CpG depleted”. For instance, a CpG-depleted dsDNA may be synthesized de novo.

Various CpG depleted molecules are provided herein. A dsDNA molecule described herein may be substantially free of (e.g., free of) CpG's. In some embodiments, the portion of the nucleotide sequence that is substantially free of (e.g., free of) CpG's is at least 10%, at least 20%, at least 30%, at least 40%, at least 50%, at least 60%, at least 70%, at least 80%, at least 85%, at least 90%, at least 95%, at least 99%, or 100% of the nucleotide sequence of the dsDNA molecule. In some embodiments, the portion of the nucleotide sequence that is substantially free of (e.g., free of) CpG's is between 10% and 20%, between 20% and 30%, between 30% and 40%, between 40% and 50%, between 50% and 60%, between 60% and 70%, between 70% and 80%, between 80% and 85%, between 85% and 90%, between 90% and 95%, between 95% and 99%, or between 99% and 100% of the nucleotide sequence of the dsDNA molecule. In some embodiments, a nucleotide sequence of a dsDNA molecule described herein comprises no more than 1, no more than 5, no more than 10, no more than 20, or no more than 50 CpG's. In some embodiments, a nucleotide sequence of a dsDNA molecule described herein comprises between 0 and 5, between 5 and 10, between 10 and 20, or between 20 and 50 CpG's. In some embodiments, a nucleotide sequence of a dsDNA molecule described herein does not comprise any CpG's.

In some embodiments, a nucleotide sequence of interest may have fewer CpG's in comparison to a reference sequence. In some embodiments, the reference sequence has at least 40%, at least 50%, at least 60%, at least 70%, at least 80%, or at least 90% sequence identity to the CpG-depleted nucleotide sequence. The reference sequence and the CpG-depleted nucleotide sequence may each comprise: an effector sequence (e.g., an effector sequence that encodes the same effector as the nucleotide sequence), a promoter sequence, an exon sequence, an intron sequence, a sequence encoding a 5′ untranslated region, a sequence encoding a 3′ untranslated region, an enhancer sequence, a sequence encoding a polyadenylation site, or any combination thereof. In some embodiments, at least 10%, at least 20%, at least 30%, at least 40%, at least 50%, at least 60%, at least 70%, at least 80%, at least 90%, at least 95%, at least 99%, or 100% of CpG's that are present in a reference sequence are absent in the CpG-depleted nucleotide sequence. In some embodiments, the effector is a naturally occurring effector. In other embodiments, the effector is not a naturally occurring effector. In some embodiments, a CpG-depleted nucleotide sequence is a non-coding sequence.

A CpG-depleted nucleotide sequence may be manually designed by a user or may be designed computationally. When designing a CpG-depleted nucleotide sequence that comprises an effector sequence encoding an effector, a user may remove one or more (e.g., all) CpG's present in a reference sequence comprising an effector sequence that encodes the same effector. The CpG-depleted nucleotide sequence may be designed, for example, using the DNA Chisel program, described in Zulkower et al., Bioinformatics, 36 (16): 4508-4509, 2020, doi.org/10.1093/bioinformatics/btaa558, which is hereby incorporated by reference in its entirety. For example, for coding regions, in some embodiments, the user begins with a reference nucleotide sequence and only changes codons that are directly affected by a CpG, e.g., by changing the codon comprising a CpG to a codon that encodes the same amino acid but lacks a CpG. Alternatively, a user may alter the sequence to remove CpG's in combination with a codon optimization strategy. In some embodiments, the codon optimization strategy is based on the codon usage for a set of reference genes. In some embodiments, the reference genes are general human genes. In some embodiments, the reference genes are T-cell specific genes. The CpG may be changed by substituting the C and/or the G.

CpG depletion can also be performed on a noncoding region. For instance, in some embodiments, one or more (e.g., substantially all, e.g., all) of the CpG's in a reference nucleotide sequence are converted to a CpA sequence to prepare the CpG-depleted nucleotide sequence. In some embodiments, one or more (e.g., substantially all, e.g., all) of the CpG's in a reference nucleotide sequence are converted to a randomized dinucleotide that is not a CpG. In some embodiments, one or more (e.g., substantially all, e.g., all) of the CpG's in a reference nucleotide sequence are converted to a dinucleotide that is not a CpG, in a way designed to minimize the impact on potential transcription factor binding sites. In some embodiments, one or more (e.g., substantially all, e.g., all) of the CpG is converted to a dinucleotide that is not a CpG, through the use of a model (e.g., a machine learning model). In some embodiments, the model is used to predict function from the nucleotide sequence. In some embodiments, the model is used to preserve one or more of the functional properties of the nucleotide sequence while simultaneously removing one or more (e.g., all) of the CpG's from the reference sequence. In some embodiments, the model used is Borzoi, as described in Linder et al., Nat Genet 57:949-961, 2025, doi.org/10.1038/s41588-024-02053-6, which is hereby incorporated by reference in its entirety. In some embodiments, the model used is dhs733, as described in Castillo-Hair et al., biorxiv, 2025, doi.org/10.1101/2025.09.30.679565, which is hereby incorporated by reference in its entirety. In some embodiments, the model used is a machine learning model trained on genetic data (e.g., publicly available Massively Parallel Reporter Assay (MPRA) data) to predict gene expression (e.g., cell-type-specific gene expression) from a nucleotide sequence.

### Structural Elements of RNA Molecules

In some embodiments, the RNA molecule described herein is linear and single stranded. In some embodiments, the RNA molecule comprises two free ends.

In some embodiments, the RNA molecule is a circular single-stranded RNA molecule, in which the RNA molecule lacks a free end. A circular RNA molecule may be covalently closed or may form a closed structure without free RNA ends through non-covalent interactions, e.g., the RNA molecule may be closed through a splint, e.g., a nucleic acid (e.g., DNA or RNA) splint, through a moiety such as a protein that binds and brings together both ends of a linear RNA molecule, or through binding of a plurality of proteins, each of two of the plurality binding to a different RNA end, and then binding to each other or a third moiety to close the RNA structure. In the case of circular RNA molecule, the term circular does not imply that the RNA structure lacks all intramolecular structure; rather, a circular RNA molecule may have short regions of intramolecular double stranded regions or other structures.

An RNA molecule being single stranded does not imply that it is entirely devoid of any intramolecular base pairing. In some embodiments, a single-stranded RNA molecule described herein may have less than a threshold level of intramolecular complementarity or double stranded structures. In one embodiment, the single stranded RNA molecule does not comprise more than 10, more than 8, more than 7, more than 5, more than 4, more than 3, more than 2, or more than 1 double stranded region longer than 20, 15, 10, or 5 base pairs. In one embodiment, the single stranded RNA molecule does not comprise more than 10, more than 8, more than 7, more than 5, more than 4, more than 3, more than 2, or more than 1 double stranded region longer than 20 base pairs. In some embodiments, the single stranded RNA molecule does not comprise any regions of intramolecular complementarity longer than 20, 15, 10, or 5 base pairs. In some embodiments, the single stranded RNA molecule does not comprise any regions of intramolecular complementarity longer than 20 base pairs. In some embodiments, the single stranded RNA molecule comprises 1, 2, 3, 4, 5, 7, 8, or 10, double stranded regions, e.g., wherein the double stranded regions are no more than 20, 15, 10, or 5 base pairs.

In some embodiments, the single-stranded RNA molecule does not form a double stranded structure longer than 20 base pairs. In some embodiments, the single-stranded RNA molecule does not comprise a first sequence that hybridizes with a second sequence, wherein the first sequence and the second sequence are at least 5, at least 10, at least 15, at least 20, or at least 25 nt long, and wherein the first sequence and the second sequence are positioned less than 6, 5, 4, 3, 2, or 1 nucleotides apart from each other.

In some embodiments, the RNA molecule is double stranded. The RNA molecule may be double stranded and circular. The RNA molecule may be double stranded and linear.

In some embodiments, the RNA molecule has a GC content of 30-40%, 40-50%, 50-60%, or 60-70%. In some embodiments, the RNA molecule lacks a viral packaging site, or wherein the RNA molecule does not encode a viral capsid gene.

In some embodiments, an RNA molecule disclosed herein is at least 50 nucleotides, at least 75 nucleotides, at least 100 nucleotides, at least 200 nucleotides, at least 300 nucleotides, at least 400 nucleotides, at least 500 nucleotides, or at least 1000 nucleotides in length. In some embodiments, the RNA molecule disclosed herein is between 40-50, 50-75, 75-100, 100-200, 200-300, 300-400, 400-500, or 500-1000 nucleotides in length. In some embodiments, the size of an RNA molecule disclosed herein is a length sufficient to encode one or more useful polypeptides.

In some embodiments, at least 80%, at least 85%, at least 90%, at least 95%, at least 98%, at least 99%, or 100% of the sugars of an RNA molecule described herein comprise-OH at their 2′ position.

In some embodiments, an RNA molecule described herein is recognized by a ribosome. In some embodiments, an RNA molecule described herein is translated, e.g., in a cell or a lysate. In an embodiment, the single stranded RNA molecule is a sense strand. In an embodiment, the single stranded RNA molecule is an antisense strand.

mRNAs

In some embodiments, an RNA molecule described herein is a messenger RNA (mRNA). Typically, an mRNA molecule has sufficient elements to be recognized by a ribosome and direct translation of a polypeptide. In some embodiments, the mRNA molecule comprises a cap. In some embodiments, the mRNA molecule comprises a polyA tail situated 3′ of the effector sequence (e.g., the region encoding the immunogen).

A 5′ cap may be canonical or chemically modified. In some embodiments, the mRNA molecule comprises one or more of: anti-reverse cap analog (ARCA; m27.3′-OGP3G), GP3G (Unmethylated Cap Analog), m7GP3G (Monomethylated Cap Analog), m32.2.7GP3G (Trimethylated Cap Analog), m5CTP (5′-methyl-cytidine triphosphate), m6ATP (N6-methyl-adenosine-5′-triphosphate), s2UTP (2-thio-uridine triphosphate), Y′ (pseudouridine triphosphate), a hypermethylated cap analog; an NAD+-derived cap analog (e.g., as described in Kiledjian, Trends in Cell Biology 28, 454-464 (2018)); or a modified, e.g., biotinylated, cap analog (e.g., as described in Bednarek et al., Phil Trans R Soc B 373, 20180167 (2018)).

In some embodiments, the mRNA molecule comprises a 7-methylguanosine cap (e.g., a O-Me-m7G cap).

In some embodiments, the mRNA molecule comprises a 3′ polyA tail. In some embodiments, the 3′ polyA tail has a length of 50-100, 100-200, or 200-300 ribonucleotides. In some embodiments, the nucleobases in the 3′ polyA tail are exclusively adenine.

In some embodiments, an mRNA molecule described herein has a sequence that is identical to a DNA molecule described herein. It is understood that the two sequences will still be considered identical even if the mRNA molecule comprises U at every position where the DNA molecule comprises T.

### Sequence Elements of RNA Molecules

In some embodiments, an RNA molecule described herein comprises an effector sequence. The effector sequence may either encode an effector (e.g., a polypeptide) or may itself be a functional RNA sequence. In some embodiments, an RNA molecule described herein comprises a heterologous functional sequence. In some embodiments, the RNA molecule comprises both of an effector sequence and a heterologous functional sequence.

In some embodiments, the effector sequence encodes a polypeptide (e.g., a protein). In some embodiments, the effector sequence is heterologous to a target cell.

The RNA molecule described herein may also include other native or heterologous expression control elements, such as a polyA tail or Kozak consensus sequences.

### Effector Sequence

The effector sequence of an RNA molecule described herein may be an RNA sequence encoding a therapeutic peptide, polypeptide or protein. In some embodiments, a polypeptide effector encoded by RNA molecule described herein is an immunogen. For example, in some embodiments, the immunogen is a viral antigen (e.g., a coronavirus antigen or a lentivirus antigen). In some embodiments, the viral antigen is a COVID-19 antigen, a hepatitis antigen (e.g., a hepatitis B antigen or a hepatitis A antigen), an influenza antigen, an HPV antigen, a chickenpox antigen, a measles antigen, a mumps antigen, a rubella antigen, or a combination thereof. In some embodiments, the immunogen is a bacterial antigen. In some embodiments, the bacterial antigen is a diphtheria antigen, a tetanus antigen, a pertussis antigen, a meningitidis antigen, a tuberculosis antigen, a cholerae antigen, or a combination thereof. In some embodiments, the immunogen is a fungal antigen. In some embodiments, the antigen is a tumor antigen.

In some embodiments, the immunogen is a peptide. In some embodiments, the immunogen polypeptide has a length of 10-20, 20-50, 50-100, 100-500, 500-1000, 1000-2000, or 2000-5000 amino acids.

In some embodiments, the immunogen is not a pneumococcal polypeptide antigen, a Bacilli antigen, a Lactobacillus antigen, a Streptococcaceae antigen, a COVID antigen, a coronavirus antigen, an HIV antigen, or a lentivirus antigen.

In embodiments, the RNA molecule can include a plurality of effector sequences. In some embodiments, the RNA molecule comprises a second effector sequence which is the same as or different than the first effector sequence. An RNA molecule can include an effector sequence that is a functional RNA and a second effector sequence that is an RNA sequence encoding a functional polypeptide. The plurality of effector sequences may be the same or different sequences of the same type.

An RNA sequence encoding a therapeutic polypeptide may be an RNA sequence encoding one or more effector which is a peptide, protein, or combinations thereof. For example, the RNA sequence is an mRNA.

In embodiments, an RNA molecule described herein may include one or a plurality of sequences encoding a polypeptide. Each of the plurality may encode the same or different protein. For example, a sequence described herein may include multiple sequences encoding multiple proteins, e.g., a plurality of proteins in a biological pathway.

In some embodiments, an RNA molecule or sequence described herein may include a plurality of sequences encoding a polypeptide, separated by a self-cleaving peptide, e.g., P2A, T2A, E2A or F2A. Self-cleaving peptides are 18-22 amino acids long, and can induce ribosomal skipping during protein translation so that two polypeptides can be encoded in the same transcript. Each of the polypeptides may encode the same or different protein. In one embodiment, an RNA molecule or sequence described herein may include a sequence encoding a first polypeptide of interest, followed by a sequence encoding a 2A self-cleaving peptide, a sequence encoding a second polypeptide of interest, and a polyA tail.

In some embodiments, the effector sequence that encodes a polypeptide or protein is codon optimized, e.g., codon optimized for expression in a mammal, e.g., a human.

### Chemically Modified Nucleotides or Ribonucleotides

The DNA molecules and the RNA molecules described herein may have chemical modifications of the nucleobases, sugars, and/or the phosphate backbone. While not wishing to be bound by theory, such modifications can be useful for protecting a DNA molecule from degradation (e.g., from exonucleases) or from the immune system of a host tissue or subject. In general, a chemically modified nucleotide or ribonucleotide has the same base-pairing specificity as the unmodified nucleotide, e.g., a chemically modified adenine “A” can base-pair with thymine “T” or uracil “U”. One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, chemical modifications (e.g., one or more modifications) are present in each of the sugar and the internucleoside linkage.

In some embodiments, an mRNA molecule described herein comprises 5-methoxyuracil.

Examples of chemical modifications to DNA useful in the methods described herein include, e.g., N6-Methyladenosine (m6A, 6 mA); 5-formylcytosine (5-formyl-2′-deoxycytosine, 5fC, f5C); 5-carboxylcytosine (5-carboxyl-2′-deoxycytosine, 5-carboxycytosine, ca5C, 5caC); 5-hydroxymethylcytosine (5-hydroxymethyl-2′-deoxycytosine, 5hmC, hm5C); 5-methyldeoxycytosine (5-methylcytosine; 5-methyl-2′-deoxycytosine; m5dC; 5mC, m5C); 5′-methylcytosine; 3-methylcytosine (m3C); 2′-fluoro-2′deoxynucleoside; 5-glucosylmethylcytosine; 5-methyl pyrimidine; 8-oxoguanine (8-oxoG); phosphorothioate; S and R phsophorothioate linkages; methylthymine; N3′-P5′ Phosphoroamidate (NP); cyclohexane nucleic acid (CeNA); tricyclo-DNA (tcDNA); or 2′-O-methyl(2′-O-Me) nucleotide. See, e.g., Pu et al. 2020. An in-vitro DNA phosphorothioate modification reaction. Mol Microbiol. 113:452-463; Zheng & Sheng. 2021. Synthesis of N4-methylcytidine (m4C) and N4,N4-dimethylcytidine (m42C) modified RNA. Current Protocols, 1, e248; Ohkubo et al. 2021. Chemical synthesis of modified oligonucleotides containing 5′-amino-5′-deoxy-5′-hydroxymethylthymidine residues. Current Protocols, 1, e70; Bao & Xu. 2021. Observation of Z-DNA structure via the synthesis of oligonucleotide DNA containing 8-trifluoromethyl-2-deoxyguanosine. Current Protocols, 1, e28; Skakujet al. 2020. Automated synthesis and purification of guanidine-backbone oligonucleotides. Current Protocols in Nucleic Acid Chemistry, 81, e110.

In some embodiments, a DNA molecule as described herein may comprise a phosphorothioate-modified nucleotide. In some embodiments, the DNA molecule described herein may include S and R phosphorothioate modified nucleotide linkages. In one embodiment, the phosphorothioate linkages are made according to Iwamoto et al, 2017, Nature Biotechnology, Volume 35:845-851. Briefly, monomers of nucleoside 3′-oxazaphospholidine derivates undergo stereocontrolled oligonucleotide synthesis with iterative capping and sulfurization to create stereocontrolled phosphorothioate linkages. The final sample is analyzed by reverse-phase high-performance liquid chromatography (RP-HPLC) and Ultraperformance liquid chromatography mass spectrometry (UPLC/MS) to determine stereochemistry of the modification. Nucleic acids containing phosphorothioate linkages are also commercially available.

In some embodiments, a DNA molecule described herein may include boranophosphate modified nucleotides, e.g., following the methods in Sergueev and Shaw, 1998, J Am Chem Soc, Volume 120, Issue 37:9417-9427. Briefly, H-phosphonate chain elongation is followed by boronation to substitute a borano group for a nonbridging oxygen in the phosphate backbone. The final sample is purified and analyzed by RP-HPLC to determine stereochemistry of the modification. Boranophosphate modified nucleotides are also commercially available.

In some embodiments, a DNA molecule described herein may include 5-methylcytosine modified nucleotides, e.g., made following the methods in Lin et al, 2002, Mol Cell Biol, Volume 22, Issue 3:704-723. Briefly, cytosine or the sequence containing cytosine is incubated with glutathione S-transferase fusion of wild-type Dnmt3a (GST-3a) protein using unlabeled S-adenosylmethionine (AdoMet). The nucleotides are purified and analyzed by HPLC to determine that the nucleotides are methylated at the correct position. 5-methylcytosine modified nucleotides are also available commercially.

In some embodiments, a DNA molecule described herein may include 7-methylguanine modified nucleotides. In one embodiment, 7-methylguanine modified nucleotides are made following the methods in Jones and Robins, 1963, Purine nucleosides. III. Methylation studies of certain naturally occurring purine nucleosides, J Am Chem Soc, Volume 85:193. Briefly, 2′-deoxyguanosine in dimethyl sulfoxide is treated with methyl iodide. The nucleotides are purified and analyzed by HPLC to determine that the nucleotides are methylated at the correct position. In another embodiment, 7-methylguanine modified nucleotides are made according to the methods described in Hendler et al, 1970, Volume 9, Issue 21:4141:4153, and Kore and Parmar, 2006, Biochemistry, Volume 25, Issue 3:337-340. Briefly, instead of guanosine 5′-diphosphate, guanine 5′-diphosphate in water is added to dimethyl sulfate to yield 7-methyl GDP. The nucleotides are purified and analyzed by HPLC to determine that the nucleotides are methylated at the correct position. 7-methylguanine modified nucleotides are also available commercially.

In some embodiments, a DNA molecule described herein comprises methylation at one or more CpG or GpC dinucleotide.

In some embodiments, a DNA molecule described herein comprises a carboxyl modification or a formyl modification.

In embodiments, chemically modified nucleotides, e.g., modifications described herein, can be introduced in the DNA molecules described herein throughout the entire sequence; within an element of a sequence, e.g., an element described herein; and/or at a 5′- or 3′-end.

In some embodiments, a chemically modified DNA molecule described herein exhibits decreased recognition by DNA sensors in a host tissue or subject compared to an unmodified DNA molecule of the same sequence, e.g., at least 10%, at least 20%, at least 30%, at least 40%, at least 50%, at least 60%, at least 70%, at least 80%, at least 90%, at least 95% or more decreased recognition by DNA sensors in a host tissue or subject compared to an unmodified DNA molecule of the same sequence. In some embodiments, a chemically modified DNA molecule described herein exhibits decreased degradation by DNA nucleases compared to an unmodified DNA molecule of the same sequence, e.g., at least 10%, at least 20%, at least 30%, at least 40%, at least 50%, at least 60%, at least 70%, at least 80%, at least 90%, at least 95% or more decreased degradation by DNA nucleases in a host tissue or subject compared to an unmodified DNA molecule. In some embodiments, a chemically modified DNA molecule described herein shows decreased activation of the innate immune system in a target/host tissue or subject compared to an unmodified DNA molecule of the same sequence, e.g., at least 10%, at least 20%, at least 30%, at least 40%, at least 50%, at least 60%, at least 70%, at least 80%, at least 90%, at least 95% or more decreased activation of the innate immune system in a target/host tissue or subject compared to an unmodified DNA molecule of the same sequence.

In some embodiments, a DNA molecule comprising chemically modified nucleotides described herein exhibits any of the following properties in a target/host tissue or subject compared to a DNA molecule of the same sequence that does not comprise chemically modified nucleotides (unmodified DNA molecule): increased integration of exogenous construct in genome of target cell; increased retention in a target cell through replication; reduced secondary or tertiary structure formation; reduced interaction with innate immune sensors; reduced interaction with nucleases; enhanced stability; enhanced longevity; reduced toxicity; enhanced delivery; increased expression; increased transport across membranes; increased binding to DNA binding moieties such as nuclear DNA binding proteins, transcription factors, chaperones, DNA polymerases. In embodiments, any of the above listed properties is modulated at least 10%, at least 20%, at least 30%, at least 40%, at least 50%, at least 60%, at least 70%, at least 80%, at least 90%, at least 95% or more in a target/host tissue or subject compared to an unmodified DNA molecule of the same sequence.

In embodiments, a DNA molecule described herein, or one strand (e.g., the sense strand or the antisense strand) of the DNA molecule, comprises between 1-100% chemically modified nucleotides, between 1%-90% chemically modified nucleotides, between 1%-80% chemically modified nucleotides, between 1%-70% chemically modified nucleotides, between 1%-60% chemically modified nucleotides, between 1%-50% chemically modified nucleotides, between 1%-40% chemically modified nucleotides, between 1%-30% chemically modified nucleotides, between 1%-20% chemically modified nucleotides, between 1%-15% chemically modified nucleotides, between 1%-10% chemically modified nucleotides, between 20%-90% chemically modified nucleotides, between 20%-80% chemically modified nucleotides. In embodiments, a DNA molecule described herein, or one strand (e.g., the sense strand or the antisense strand) of the DNA molecule, comprises at least 1% chemically modified nucleotides, at least 5% chemically modified nucleotides; at least 10% chemically modified nucleotides; at least 15% chemically modified nucleotides; at least 20% chemically modified nucleotides; at least 25% chemically modified nucleotides; at least 30% chemically modified nucleotides; at least 40% chemically modified nucleotides; at least 50% chemically modified nucleotides; at least 60% chemically modified nucleotides; at least 70% chemically modified nucleotides; at least 80% chemically modified nucleotides; at least 85% chemically modified nucleotides; at least 90% chemically modified nucleotides; at least 92% chemically modified nucleotides; at least 95% chemically modified nucleotides; at least 97% chemically modified nucleotides. In embodiments, a DNA molecule described herein, or one strand (e.g., the sense strand or the antisense strand) of the DNA molecule, comprises chemically modified nucleotides at between 0%-100% of each distinct nucleotide, e.g., 0%-100% chemically modified T nucleotides, 0%-100% chemically modified A nucleotides, 0%-100% chemically modified C nucleotides, and 0%-100% chemically modified G nucleotides for each construct. In embodiments, a DNA molecule described herein, or one strand (e.g., the sense strand or the antisense strand) of the double-stranded DNA molecule, comprises chemically modified nucleotides at between 0-100%, 10%-100%, 20%-100%, 30%-100%, 40%-100%, 50%-100%, 60%-100%, 10%-50% of each distinct nucleotide, e.g., between 0-100%, 10%-100%, 20%-100%, 30%-100%, 40%-100%, 50%-100%, 60%-100%, 10%-50% of chemically modified T nucleotides; between 0-100%, 10%-100%, 20%-100%, 30%-100%, 40%-100%, 50%-100%, 60%-100%, 10%-50% of chemically modified A nucleotides; between 0-100%, 10%-100%, 20%-100%, 30%-100%, 40%-100%, 50%-100%, 60%-100%, 10%-50% of chemically modified C nucleotides; or between 0-100%, 10%-100%, 20%-100%, 30%-100%, 40%-100%, 50%-100%, 60%-100%, 10%-50% of chemically modified G nucleotides. For example, a DNA molecule could contain 100% chemically modified T nucleotides, 50% chemically modified A nucleotides, 0% chemically modified C nucleotides, and 25% chemically modified G nucleotides.

In some embodiments, a double-stranded DNA molecule as described herein comprises chemically modified nucleotides on both strands. In certain embodiments, both strands comprise chemical modifications at the same positions (e.g., chemically modified nucleotides on one strand are base-paired with chemically modified nucleotides on the opposite strand, and/or non-chemically modified nucleotides on one strand are base-paired with non-chemically modified nucleotides on the opposite strand). In embodiments, the entirety of both strands are composed of chemically modified nucleotides. In other embodiments, the two strands of a double-stranded DNA molecule as described herein comprise different chemical modification patterns (e.g., one or more chemically modified nucleotides on one strand are base-paired with non-chemically modified nucleotides on the other strand). In embodiments, a double-stranded DNA molecule as described herein comprises one or more double-stranded regions in which both strands are chemically modified, and/or one or more double-stranded regions in which neither strand is chemically modified. In embodiments, a double-stranded DNA molecule as described herein comprises one or more double-stranded regions in which one strand is chemically modified and the other is not.

In some embodiments, a DNA molecule described herein comprises a chemically modified nucleobase having one or more structural differences relative to the canonical nucleobases (i.e., guanine, thymine, cytosine, and adenine). A chemically modified nucleobase may be produced directly by chemical synthesis, or by covalently modifying a canonical nucleobase. In some embodiments, the chemically modified nucleobase in the dsDNA region is a canonical uracil nucleobase.

In some embodiments, a DNA molecule described herein comprises a chemically modified cytosine nucleobase. In some embodiments, the chemically modified cytosine nucleobase comprises a substitution other than hydrogen at the carbon 5 (C-5) position of the nucleobase. In some embodiments, the chemically modified cytosine nucleobase comprises the

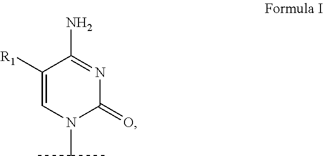

wherein R₁ is selected from the group consisting of —OH; -aldehyde; -carboxylic acid; -alkyl; —(CH₂)ₘOR₂, m=1-3 and R₂═H or a sugar molecule; and -propargylamino. In some embodiments, R₁ is selected from the group consisting of —OH; —CHO; —COOH; -alkyl; —(CH₂)ₘOR₂, m=1-3 and R₂═H or a sugar molecule; and -propargylamino, wherein the alkyl group includes one to six carbons. In some embodiments, R₁ is selected from the group consisting of —OH; —CHO; —COOH; —CH₂OR₃, R₃═H or glucose; -methyl; and -propargylamino. In some embodiments, the chemically modified cytosine nucleobase comprises 5-formylcytosine, 5-hydroxycytosine, 5-carboxycytosine, 5-propargylaminocytosine, 5-methylcytosine, 5-hydroxymethylcytosine, or glucosyl-5-hydroxymethylcytosine. Chemically modified cytosine nucleobases are further described in International Application WO/2024/173836, which is herein incorporated by reference in its entirety.

In some embodiments, a DNA molecule described herein comprises a chemically modified uracil nucleobase. In some embodiments, the chemically modified uracil nucleobase comprises a substitution other than hydrogen or a methyl group at the carbon 5 (C-5) position of the nucleobase. In some embodiments, the chemically modified uracil nucleobase comprises the structure of Formula II:

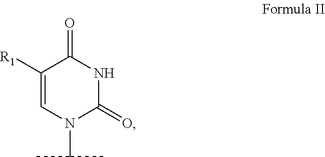

wherein R₁ is selected from the group consisting of —(CH₂)ₘOH, m=1-10; -halogen; —(CH₂)ₙ—CHO, n=0-10; —(CH₂)ₚCOOH, p=0-10; -aminoallyl; —S—(C1-C6)alkyl; and -propargylamino. In some embodiments, R₁ is selected from the group consisting of —(CH₂)ₘOH, m=1-6; -halogen; —(CH₂)ₙ—CHO, n=0-6; —(CH₂)ₚCOOH, p=0-6; -aminoallyl; —S—(C1-C3)alkyl; and -propargylamino. In some embodiments, R₁ is selected from the group consisting of —(CH₂)OH; —I; —Br; —CHO; —COOH; -aminoallyl; —S-methyl; and -propargylamino. In some embodiments, the chemically modified uracil nucleobase comprises 5-hydroxymethyluracil, 5-aminoallyluracil, 5-bromouracil, 5-iodouracil, 5-propargylaminouracil, 5-formyluracil, 5-carboxyuracil, or 5-methylthiouracil. Chemically modified uracil nucleobases are further described in International Application WO/2024/173828, which is herein incorporated by reference in its entirety.

In some embodiments, a DNA molecule described herein comprises a first type of chemically modified nucleobase and a second type of chemically modified nucleobase. In some embodiments, the first type of chemically modified nucleobase is a chemically modified cytosine nucleobase and the second type of chemically modified nucleobase is a chemically modified uracil nucleobase. In some embodiments, the first type of chemically modified nucleobase is a chemically modified cytosine nucleobase and the second type of chemically modified nucleobase is a different chemically modified cytosine nucleobase. In some embodiments, the first type of chemically modified nucleobase is a chemically modified uracil nucleobase and the second type of chemically modified nucleobase is a different chemically modified uracil nucleobase.

### Asymmetrically Modified DNA Molecules

In some embodiments, a DNA molecule described herein (e.g., a double stranded DNA molecule described herein, e.g., a linear dsDNA molecule comprising two closed ends described herein, or a circular double-stranded DNA molecule described herein) comprises a sense strand and an antisense strand. In some embodiments, the antisense strand comprises one or more chemically modified nucleotides. In some embodiments, the antisense strand is substantially free of (e.g., is free of) chemically modified nucleotides. In some embodiments, the sense strand comprises one or more chemically modified nucleotides. In some embodiments, the sense strand is substantially free of (e.g., is free of) chemically modified nucleotides. In some embodiments, the sense strand comprises one or more chemically modified nucleotides, and the antisense strand is substantially free of (e.g., is free of) chemically modified nucleotides. In some embodiments, the chemically modified nucleotide comprises a backbone modification (e.g., a phosphorothioate linkage). In some embodiments, the chemically modified nucleotide comprises a chemically modified sugar. In some embodiments, the chemically modified nucleotide comprises a chemically modified nucleobase.

In some embodiments, the antisense strand comprises one or more chemically modified nucleobases. In some embodiments, the antisense strand is substantially free of (e.g., is free of) chemically modified nucleobases. In some embodiments, the sense strand comprises one or more chemically modified nucleobases. In some embodiments, the sense strand is substantially free of (e.g., is free of) chemically modified nucleobases.

In some embodiments, the sense strand comprises one or more chemically modified nucleobases, and the antisense strand is substantially free of (e.g., is free of) chemically modified nucleobases. In some embodiments, at least 1%, at least 5%, at least 10%, at least 15%, at least 20%, at least 25%, at least 30%, at least 35%, at least 40%, at least 45%, or at least 50% of positions in the sense strand of the DNA molecule comprise chemically modified nucleobases. In some embodiments, at least 10% of positions in the sense strand of the DNA molecule comprise chemically modified nucleobases, and the antisense strand is free of chemically modified nucleobases. In some embodiments, at least 20% of positions in the sense strand of the DNA molecule comprise chemically modified nucleobases, and the antisense strand is free of chemically modified nucleobases. In some embodiments, at least 30% of positions in the sense strand of the DNA molecule comprise chemically modified nucleobases, and the antisense strand is free of chemically modified nucleobases. In some embodiments, at least 40% of positions in the sense strand of the DNA molecule comprise chemically modified nucleobases, and the antisense strand is free of chemically modified nucleobases. In some embodiments, 1%-50% (e.g., 1%-5%, 5%-10%, 10-15%, 15%-20%, 20%-25%, 25%-30%, 30%-35%, 35%-40%, 40%-45%, or 45%-50%) of positions in the sense strand of the DNA molecule comprise chemically modified nucleobases. In some embodiments, 10%-20% of positions in the sense strand of the DNA molecule comprise chemically modified nucleobases, and the antisense strand is free of chemically modified nucleobases. In some embodiments, 20%-30% of positions in the sense strand of the DNA molecule comprise chemically modified nucleobases, and the antisense strand is free of chemically modified nucleobases. In some embodiments, 30%-50% of positions in the sense strand of the DNA molecule comprise chemically modified nucleobases, and the antisense strand is free of chemically modified nucleobases. In some embodiments, at least 1%, at least 5%, at least 10%, at least 15%, at least 20%, or at least 25% of positions in the sense strand of the DNA molecule comprise the same chemically modified nucleobase. In some embodiments, 1%-25% (e.g., 1%-5%, 5%-10%, 10%-15%, 15%-20%, or 20%-25%) of positions in the sense strand of the DNA molecule comprise the same chemically modified nucleobase. In some embodiments, the antisense strand comprises no more than 10, no more than 9, no more than 8, no more than 7, no more than 6, no more than 5, no more than 4, no more than 3, no more than 2, or no more than 1 chemically modified nucleotides. In some embodiments, the antisense strand comprises no more than 10, no more than 9, no more than 8, no more than 7, no more than 6, no more than 5, no more than 4, no more than 3, no more than 2, or no more than 1 chemically modified nucleobases.

In some embodiments, the DNA molecule comprises an upstream DNA end form which is a closed end, a double stranded region comprising a sense strand and an antisense strand, and a downstream DNA end form which is a closed end, wherein the sense strand comprises one or more chemically modified nucleobases, and wherein the antisense strand is substantially free of (e.g., is free of) chemically modified nucleobases. In some embodiments, the sense strand comprises one or more (e.g., at least 3) backbone modifications, e.g., phosphorothioate linkages. In some embodiments, the antisense strand comprises one or more (e.g., at least 3) backbone modifications, e.g., phosphorothioate linkages. In some embodiments, the one or more backbone modifications are situated between the 2, 3, 4, 5, 6, 7, 8, 9, or 10 nucleotides adjacent to the upstream DNA end form and/or downstream DNA end form. In some embodiments, the sense strand comprises a region that is substantially free of (e.g., is free of) backbone modifications. In some embodiments, the region is at least 50, at least 100, at least 200, at least 300, at least 400, at least 500, or at least 1,000 nucleotides in length. In some embodiments, the region is 50-100, 100-200, 200-300, 300-400, 400-500, 500-600, 600-700, 700-800, 800-900, 900-1000, 1000-1500, or 1500-2000 nucleotides in length.

In some embodiments, the longest stretch of unmodified nucleotides in the sense strand is no more than 1000, no more than 900, no more than 800, no more than 700, no more than 600, no more than 500, no more than 400, no more than 300, no more than 200, no more than 100, no more than 50, or no more than 10 nucleotides. In some embodiments, the longest stretch of unmodified nucleotides in the sense strand is 10-50, 50-100, 100-200, 200-300, 300-400, 400-500, 500-600, 600-700, 700-800, 800-900, or 900-1000 nucleotides. In some embodiments, the longest stretch of unmodified nucleobases in the sense strand is no more than 1000, no more than 900, no more than 800, no more than 700, no more than 600, no more than 500, no more than 400, no more than 300, no more than 200, no more than 100, no more than 50, or no more than 10 nucleobases. In some embodiments, the longest stretch of unmodified nucleobases in the sense strand is 10-50, 50-100, 100-200, 200-300, 300-400, 400-500, 500-600, 600-700, 700-800, 800-900, or 900-1000 nucleobases. In some embodiments, the longest stretch of base pairs that is substantially free of (e.g., is free of) chemically modified nucleobases in the DNA molecule is no longer than 40, no longer than 30, no longer than 20, or no longer than 10 base pairs.

In some embodiments, the sense strand comprises a uracil nucleobase. In some embodiments, at least 1%, at least 5%, at least 10%, at least 15%, at least 20%, at least 25%, at least 50%, at least 75%, at least 90%, at least 95%, at least 96%, at least 97%, at least 98%, at least 99%, or all of thymine or uracil positions in the sense strand of the DNA molecule comprise a uracil nucleobase. In some embodiments, at least 20% of thymine or uracil positions in the sense strand of the DNA molecule comprise a uracil nucleobase, and the antisense strand is free of chemically modified nucleobases. In some embodiments, at least 40% of thymine or uracil positions in the sense strand of the DNA molecule comprise a uracil nucleobase, and the antisense strand is free of chemically modified nucleobases. In some embodiments, at least 80% of thymine or uracil positions in the sense strand of the DNA molecule comprise a uracil nucleobase, and the antisense strand is free of chemically modified nucleobases. In some embodiments, 1%-100% (e.g., 1%-5%, 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, 95%-96%, 96%-97%, 97%-98%, 98%-99%, 99%-100%, or 95%-100%) of thymine or uracil positions in the sense strand of the DNA molecule comprise a uracil nucleobase. In some embodiments, every thymine or uracil position in a stretch of at least 100, at least 200, at least 300, at least 400, at least 500, at least 600, at least 700, at least 800, at least 900, at least 1000, at least 1500, or at least 2000 nucleotides in the sense strand of the DNA molecule comprises a uracil nucleobase. In some embodiments, every thymine or uracil position in a stretch of 100-200, 200-300, 300-400, 400-500, 500-600, 600-700, 700-800, 800-900, 900-1000, 1000-1500, or 1500-2000 nucleotides in the sense strand of the DNA molecule comprises a uracil nucleobase. In some embodiments, the uracil nucleobase is a canonical uracil nucleobase. In some embodiments, the uracil nucleobase is a chemically modified uracil nucleobase. In some embodiments, the chemically modified uracil nucleobase comprises 5-hydroxymethyluracil.

In some embodiments, the sense strand comprises a chemically modified cytosine nucleobase. In some embodiments, at least 1%, at least 5%, at least 10%, at least 15%, at least 20%, at least 25%, at least 50%, at least 75%, at least 90%, at least 95%, at least 96%, at least 97%, at least 98%, at least 99%, or all of cytosine positions in the sense strand of the DNA molecule comprise a chemically modified cytosine nucleobase. In some embodiments, at least 20% of cytosine positions in the sense strand of the DNA molecule comprise a chemically modified cytosine nucleobase, and the antisense strand is free of chemically modified nucleobases. In some embodiments, at least 40% of cytosine positions in the sense strand of the DNA molecule comprise a chemically modified cytosine nucleobase, and the antisense strand is free of chemically modified nucleobases. In some embodiments, at least 80% of cytosine positions in the sense strand of the DNA molecule comprise a chemically modified cytosine nucleobase, and the antisense strand is free of chemically modified nucleobases. In some embodiments, 1%-100% (e.g., 1%-5%, 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, 95%-96%, 96%-97%, 97%-98%, 98%-99%, 99%-100%, or 95%-100%) of cytosine positions in the sense strand of the DNA molecule comprise a chemically modified cytosine nucleobase. In some embodiments, every cytosine position in a stretch of at least 100, at least 200, at least 300, at least 400, at least 500, at least 600, at least 700, at least 800, at least 900, at least 1000, at least 1500, or at least 2000 nucleotides in the sense strand of the DNA molecule comprises a chemically modified cytosine nucleobase. In some embodiments, every cytosine position in a stretch of 100-200, 200-300, 300-400, 400-500, 500-600, 600-700, 700-800, 800-900, 900-1000, 1000-1500, or 1500-2000 nucleotides in the sense strand of the DNA molecule comprises a chemically modified cytosine nucleobase. In some embodiments, the chemically modified cytosine nucleobase comprises 5-hydroxycytosine.

In some embodiments, the sense strand comprises a chemically modified guanine nucleobase. In some embodiments, at least 1%, at least 5%, at least 10%, at least 15%, at least 20%, at least 25%, at least 50%, or at least 75% of guanine positions in the sense strand of the DNA molecule comprise a chemically modified guanine nucleobase. In some embodiments, at least 40% of guanine positions in the sense strand of the DNA molecule comprise a chemically modified guanine nucleobase, and the antisense strand is free of chemically modified nucleobases. In some embodiments, 1%-75% (e.g., 1%-5%, 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, or 70%-75%) of guanine positions in the sense strand of the DNA molecule comprise a chemically modified guanine nucleobase. In some embodiments, every guanine position in a stretch of at least 100, at least 200, at least 300, at least 400, at least 500, at least 600, at least 700, at least 800, at least 900, at least 1000, at least 1500, or at least 2000 nucleotides in the sense strand of the DNA molecule comprises a chemically modified guanine nucleobase. In some embodiments, every guanine position in a stretch of 100-200, 200-300, 300-400, 400-500, 500-600, 600-700, 700-800, 800-900, 900-1000, 1000-1500, or 1500-2000 nucleotides in the sense strand of the DNA molecule comprises a chemically modified guanine nucleobase. In some embodiments, the chemically modified guanine nucleobase comprises inosine.

In some embodiments, a DNA molecule described herein comprises a sense strand and an antisense strand, wherein the sense strand comprises one or more chemically modified nucleobases, and the antisense strand is substantially free of (e.g., is free of) chemically modified nucleobases. In some embodiments, the DNA molecule, when contacted to human cells, results in a lower level of IL6 or CXCL10 mRNA compared to a control DNA molecule (e.g., lower by at least 10%, at least 20%, at least 30%, at least 40%, or at least 50%), wherein the control DNA molecule comprises the same sequence and same double stranded form as the DNA molecule, but comprises no chemically modified nucleobases. In some embodiments, the DNA molecule, when contacted to human cells, results in expression at a level at least 80%, at least 90%, at least 100%, at least 120%, at least 140%, at least 160%, or at least 180% the expression of an unmodified control DNA molecule, wherein the unmodified control DNA molecule comprises the same sequence and same double stranded form as the DNA molecule, but comprises no chemically modified nucleobases. In some embodiments, the DNA molecule, when contacted to human cells, results in expression at a level at least the expression of a modified control DNA molecule, wherein the modified control DNA molecule comprises the same sequence, same double stranded form, and same degree of sense strand nucleobase modification as the DNA molecule, but comprises antisense strand nucleobase modification at the same degree as sense strand nucleobase modification.

In some embodiments, a DNA molecule described herein comprises a first type of chemically modified nucleobase and a second type of chemically modified nucleobase. In some embodiments, the DNA molecule comprises a sense strand and an antisense strand, wherein the sense strand comprises a first type of chemically modified nucleobase and a second type of chemically modified nucleobase. In some embodiments, the antisense strand is substantially free of (e.g., is free of) chemically modified nucleobases. In some embodiments, the first type of chemically modified nucleobase is a chemically modified cytosine nucleobase, and the second type of chemically modified nucleobase is a different chemically modified cytosine nucleobase, a uracil nucleobase, or a chemically modified guanine nucleobase. In some embodiments, the first type of chemically modified nucleobase is a uracil nucleobase, and the second type of chemically modified nucleobase is a chemically modified cytosine nucleobase, a different uracil nucleobase, or a chemically modified guanine nucleobase.

### Asymmetrically Modified Circular DNA Molecules

In some embodiments, a composition described herein comprises, or a method described herein comprises the use of, a double stranded DNA molecule (dsDNA molecule), wherein the dsDNA molecule is circular, and the dsDNA molecule comprises a first strand (e.g., sense strand) and a second strand (e.g., an antisense strand), wherein the first strand comprises one or more chemically modified nucleobases, and the second strand is substantially free of (e.g., is free of) chemically modified nucleobases. In some embodiments, the first strand is a sense strand and the second strand is an antisense strand. In some embodiments, the first strand is an antisense strand and the second strand is a sense strand.

In some embodiments, the dsDNA molecule comprises a promoter sequence and an effector sequence that encodes an effector (e.g., a therapeutic effector). In some embodiments, the dsDNA molecule comprises one or more sequences encoding a 5′ untranslated region (5′ UTR) that is 5′ of the effector sequence and/or a 3′ untranslated region (3′ UTR) that is 3′ of the effector sequence. In some embodiments, the dsDNA molecule comprises a sequence encoding a polyadenylation site. In some embodiments, the dsDNA molecule comprises an intron sequence. In some embodiments, the dsDNA molecule comprises an enhancer sequence. In some aspects, one or more regions of a dsDNA molecule described herein comprises a first strand and a second strand, wherein the first strand of said one or more regions comprises one or more chemically modified nucleobases, and the second strand of said one or more regions is substantially free of (e.g., is free of) chemically modified nucleobases. In some embodiments, the dsDNA molecule comprises two or more such regions. In some embodiments, the first strand of the first region is contiguous with the first strand of the second region. In some embodiments, the first strand of the first region is contiguous with the second strand of the second region. In some embodiments, the entire length of the dsDNA molecule is made up of such regions.

In some embodiments, the first strand (e.g., sense strand) comprises one or more backbone modifications, e.g., phosphorothioate linkages. In some embodiments, the second strand (e.g., antisense strand) is substantially free of (e.g., is free of) phosphorothioate linkages. In some embodiments, the second strand (e.g., antisense strand) is substantially free of (e.g., is free of) backbone modifications.

In some embodiments, at least 1%, at least 5%, at least 10%, at least 15%, at least 20%, at least 25%, at least 30%, at least 35%, at least 40%, at least 45%, or at least 50% positions in the first strand (e.g., sense strand) of the dsDNA molecule comprise chemically modified nucleobases. In some embodiments, at least 1%, at least 5%, at least 10%, at least 15%, at least 20%, or at least 25% positions in the first strand (e.g., sense strand) of the dsDNA molecule comprise the same chemically modified nucleobase. In some embodiments, 1%-50% (e.g., 1%-25%, 1%-5%, 5%-10%, 10%-15%, 15%-20%, 20%-25%, 25%-30%, 30%-35%, 35%-40%, 40%-45%, or 45%-50%) positions in the first strand (e.g., sense strand) of the dsDNA molecule comprise chemically modified nucleobases. In some embodiments, 1%-25% (e.g., 1%-5%, 5%-10%, 10%-15%, 15%-20%, or 20%-25%) positions in the first strand (e.g., sense strand) of the dsDNA molecule comprise the same chemically modified nucleobase. In some embodiments, at least 80%, at least 85%, at least 90%, at least 95%, or all the chemically modified nucleobases of the dsDNA molecule have the same chemical structure. In some embodiments, 80%-85%, 85%-90%, 90%-95%, or 95%-100% of the chemically modified nucleobases of the dsDNA molecule have the same chemical structure.

In some embodiments, the chemically modified nucleobase comprises a uracil nucleobase. In some embodiments, the uracil nucleobase is a canonical uracil nucleobase or a chemically modified uracil nucleobase. In some embodiments, the uracil nucleobase is a canonical uracil nucleobase. In some embodiments, the uracil nucleobase is a chemically modified uracil nucleobase. In some embodiments, at least 1%, at least 5%, at least 10%, at least 15%, at least 20%, at least 25%, at least 50%, at least 75%, at least 90%, at least 95%, at least 96%, at least 97%, at least 98%, or at least 99% of thymine or uracil positions in the first strand (e.g., sense strand) of the dsDNA molecule comprise a uracil nucleobase. In some embodiments, 1%-100% (e.g., 1%-5%, 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, 95%-96%, 96%-97%, 97%-98%, 98%-99%, or 95%-100%) of thymine or uracil positions in the first strand (e.g., sense strand) of the dsDNA molecule comprise a uracil nucleobase. In some embodiments, at least 20% of thymine or uracil positions in the first strand (e.g., sense strand) of the dsDNA molecule comprise a uracil nucleobase. In some embodiments, at least 40% of thymine or uracil positions in the first strand (e.g., sense strand) of the dsDNA molecule comprise a uracil nucleobase. In some embodiments, at least 80% of thymine or uracil positions in the first strand (e.g., sense strand) of the dsDNA molecule comprise a uracil nucleobase. In some embodiments, every thymine or uracil position in a stretch of at least 100, at least 200, at least 300, at least 400, at least 500, at least 600, at least 700, at least 800, at least 900, at least 1000, at least 1500, or at least 2000 nucleotides in the first strand (e.g., sense strand) of the dsDNA molecule comprises a uracil nucleobase. In some embodiments, every thymine or uracil position in a stretch of 100-200, 200-300, 300-400, 400-500, 500-600, 600-700, 700-800, 800-900, 900-1000, 1000-1500, or 1500-2000 nucleotides in the first strand (e.g., sense strand) of the dsDNA molecule comprises a uracil nucleobase. In some embodiments, the chemically modified nucleobase comprises a chemically modified uracil nucleobase. In some embodiments, the chemically modified uracil nucleobase comprises 5-hydroxymethyluracil. In some embodiments, at least 80%, at least 85%, at least 90%, at least 95%, or all of the chemically modified nucleobases of the dsDNA molecule comprise 5-hydroxymethyluracil. In some embodiments, 80%-85%, 85%-90%, 90%-95%, or 95%-100% of the chemically modified nucleobases of the dsDNA molecule comprise 5-hydroxymethyluracil.

In some embodiments, the chemically modified nucleobase comprises a canonical uracil nucleobase. In some embodiments, at least 80%, at least 85%, at least 90%, at least 95%, or all of the chemically modified nucleobases of the dsDNA molecule comprise a canonical uracil nucleobase. In some embodiments, 80%-85%, 85%-90%, 90%-95%, or 95%-100% of the chemically modified nucleobases of the dsDNA molecule comprise a canonical uracil nucleobase.

In some embodiments, the chemically modified nucleobase comprises a chemically modified cytosine nucleobase. In some embodiments, at least 1%, at least 5%, at least 10%, at least 15%, at least 20%, at least 25%, at least 50%, at least 75%, at least 90%, at least 95%, at least 96%, at least 97%, at least 98%, at least 99%, or all of cytosine positions in the first strand (e.g., sense strand) of the dsDNA molecule comprise a chemically modified cytosine nucleobase. In some embodiments, 1%-100% (e.g., 1%-5%, 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, 95%-96%, 96%-97%, 97%-98%, 98%-99%, 99%-100%, or 95%-100%) of cytosine positions in the first strand (e.g., sense strand) of the dsDNA molecule comprise a chemically modified cytosine nucleobase. In some embodiments, at least 20% of cytosine positions in the first strand (e.g., sense strand) of the dsDNA molecule comprise a chemically modified cytosine nucleobase. In some embodiments, at least 40% of cytosine positions in the first strand (e.g., sense strand) of the dsDNA molecule comprise a chemically modified cytosine nucleobase. In some embodiments, at least 80% of cytosine positions in the first strand (e.g., sense strand) of the dsDNA molecule comprise a chemically modified cytosine nucleobase. In some embodiments, every cytosine position in a stretch of at least 100, at least 200, at least 300, at least 400, at least 500, at least 600, at least 700, at least 800, at least 900, at least 1000, at least 1500, or at least 2000 nucleotides in the first strand (e.g., sense strand) of the dsDNA molecule comprises a chemically modified cytosine nucleobase. In some embodiments, every cytosine position in a stretch of 100-200, 200-300, 300-400, 400-500, 500-600, 600-700, 700-800, 800-900, 900-1000, 1000-1500, or 1500-2000 nucleotides in the first strand (e.g., sense strand) of the dsDNA molecule comprises a chemically modified cytosine nucleobase. In some embodiments, the chemically modified cytosine nucleobase comprises 5-hydroxycytosine.

In some embodiments, the chemically modified nucleobase comprises a chemically modified guanine nucleobase. In some embodiments, at least 1%, at least 5%, at least 10%, at least 15%, at least 20%, at least 25%, at least 50%, or at least 75% of guanine positions in the first strand (e.g., sense strand) of the dsDNA molecule comprise a chemically modified guanine nucleobase. In some embodiments, 1%-75% (e.g., 1%-5%, 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, or 70%-75%) of guanine positions in the first strand (e.g., sense strand) of the dsDNA molecule comprise a chemically modified guanine nucleobase. In some embodiments, at least 40% of guanine positions in the first strand (e.g., sense strand) of the dsDNA molecule comprise a chemically modified guanine nucleobase. In some embodiments, every guanine position in a stretch of at least 100, at least 200, at least 300, at least 400, at least 500, at least 600, at least 700, at least 800, at least 900, at least 1000, at least 1500, or at least 2000 nucleotides in the first strand (e.g., sense strand) of the dsDNA molecule comprises a chemically modified guanine nucleobase. In some embodiments, every guanine position in a stretch of 100-200, 200-300, 300-400, 400-500, 500-600, 600-700, 700-800, 800-900, 900-1000, 1000-1500, or 1500-2000 nucleotides in the first strand (e.g., sense strand) of the dsDNA molecule comprises a chemically modified guanine nucleobase. In some embodiments, the chemically modified guanine nucleobase comprises inosine.

In some embodiments, the first strand of the dsDNA molecule comprises a first type of chemically modified nucleobase and a second type of chemically modified nucleobase. In some embodiments, the first type of chemically modified nucleobase is a chemically modified cytosine nucleobase, and the second type of chemically modified nucleobase is a different chemically modified cytosine nucleobase, a uracil nucleobase, or a chemically modified guanine nucleobase. In some embodiments, the first type of chemically modified nucleobase is a uracil nucleobase, and the second type of chemically modified nucleobase is a chemically modified cytosine nucleobase, a different uracil nucleobase, or a chemically modified guanine nucleobase. In some embodiments, the first type of chemically modified nucleobase is a chemically modified guanine nucleobase, and the second type of chemically modified nucleobase is a chemically modified cytosine nucleobase or a uracil nucleobase. In some embodiments, the chemically modified guanine nucleobase comprises inosine. In some embodiments, the uracil nucleobase comprises a canonical uracil nucleobase. In some embodiments, the uracil nucleobase comprises 5-hydroxymethyluracil. In some embodiments, the chemically modified cytosine nucleobase comprises 5-hydroxycytosine.

Chemically modified cytosine nucleobases are further described in International Application WO/2024/173836, which is herein incorporated by reference in its entirety. Chemically modified uracil nucleobases are further described in International Application WO/2024/173828, which is herein incorporated by reference in its entirety.

In some embodiments, the longest stretch of unmodified nucleotides in the first strand (e.g., sense strand) is no more than 1000, no more than 900, no more than 800, no more than 700, no more than 600, no more than 500, no more than 400, no more than 300, no more than 200, no more than 100, no more than 50, or no more than 10 nucleotides. In some embodiments, the longest stretch of unmodified nucleotides in the first strand (e.g., sense strand) is 10-50, 50-100, 100-200, 200-300, 300-400, 400-500, 500-600, 600-700, 700-800, 800-900, or 900-1000 nucleotides. In some embodiments, the longest stretch of unmodified nucleobases in the first strand (e.g., sense strand) is no more than 1000, no more than 900, no more than 800, no more than 700, no more than 600, no more than 500, no more than 400, no more than 300, no more than 200, no more than 100, no more than 50, or no more than 10 nucleobases. In some embodiments, the longest stretch of unmodified nucleobases in the first strand (e.g., sense strand) is 10-50, 50-100, 100-200, 200-300, 300-400, 400-500, 500-600, 600-700, 700-800, 800-900, or 900-1000 nucleobases. In some embodiments, the second strand (e.g., antisense strand) comprises no more than 10, no more than 9, no more than 8, no more than 7, no more than 6, no more than 5, no more than 4, no more than 3, no more than 2, or no more than 1 chemically modified nucleotides. In some embodiments, the second strand (e.g., antisense strand) comprises no more than 10, no more than 9, no more than 8, no more than 7, no more than 6, no more than 5, no more than 4, no more than 3, no more than 2, or no more than 1 chemically modified nucleobases. In some embodiments, the second strand (e.g., antisense strand) comprises no more than 10, no more than 9, no more than 8, no more than 7, no more than 6, no more than 5, no more than 4, no more than 3, no more than 2, or no more than 1 backbone modifications. In some embodiments, the second strand (e.g., antisense strand) comprises no more than 10, no more than 9, no more than 8, no more than 7, no more than 6, no more than 5, no more than 4, no more than 3, no more than 2, or no more than 1 nucleotides having a chemically modified sugar.

In some embodiments, the dsDNA molecule further comprises one or more additional chemically modified nucleotide, wherein the additional chemically modified nucleotide comprises a modification in the backbone, sugar, or nucleobase. In some embodiments, one or more of the chemically modified nucleotides is conjugated to a peptide or protein. In some embodiments, one or more of the chemically modified nucleotides comprises a phosphorothioate linkage. In some embodiments, each of the first strand (e.g., sense strand) and second strand (e.g., antisense strand) of the dsDNA molecule comprises one or more chemically modified nucleotides. In some embodiments, each of the first strand (e.g., sense strand) and second strand (e.g., antisense strand) of the dsDNA molecule comprises one or more phosphorothioate linkages.

In some aspects, the present disclosure provides a composition, e.g., a pharmaceutical composition, comprising the dsDNA molecule. In some aspects, the present disclosure provides a composition, e.g., a pharmaceutical composition, comprising a plurality of the dsDNA molecules.

Chemically modified DNA molecules, compositions comprising said DNA molecules, methods of making and using such compositions are further described in International Application WO/2026/006577, which is herein incorporated by reference in its entirety.

### Production

In some embodiments, a composition (e.g., a pharmaceutical composition) described herein comprises an ionizable lipid encapsulating one or both of a circular dsDNA molecule (cdsDNA) or a linear mRNA molecule. In some embodiments, the composition is produced by diluting the DNA molecule and/or mRNA molecule into a buffer (e.g., a citrate buffer), and diluting a lipid mix (e.g. a lipid mix comprising 50% ionizable lipid, 10% DSPC, 38.5% Cholesterol and 1.5% DMG-PEG2000 by mole) dissolved into ethanol. In some embodiments, the two solutions are mixed at a vol:vol ratio of about 3:1 (mRNA, DNA, or the combination of mRNA and DNA:lipids).

In some aspects, the present disclosure provides a method that comprises making or manufacturing a DNA molecule or an mRNA molecule, the method comprising (a) providing a DNA molecule or the mRNA molecule described herein, and (b) determining whether the structure of the DNA molecule or the mRNA molecule matches a reference structure, thereby making or manufacturing the DNA molecule or the mRNA molecule. In some embodiments, the determining of (b) comprises sequencing the DNA molecule or the mRNA molecule. In some embodiments, the determining of (b) comprises digesting the DNA molecule or the mRNA molecule with a restriction enzyme. In some embodiments, the structure of the DNA molecule or the mRNA molecule that matches the reference structure is identical to the reference structure. In some embodiments, the structure of the DNA molecule or the mRNA molecule that matches the reference structure has the same sequence as the reference structure. In some embodiments, the structure of the DNA molecule or the mRNA molecule that matches the reference structure has the same length as the reference structure.

A composition described herein may be enriched or purified from impurities or byproducts selected from the group consisting of: endotoxin, mononucleotides, chemically modified mononucleotides, single stranded DNA, proteins (e.g., enzymes, e.g., ligases, restriction enzymes), and DNA fragments or truncations. In some embodiments, a composition described herein is substantially free of process byproducts and impurities, e.g., process byproducts or impurities described herein.

In some embodiments, a composition described herein is formulated with a lipid-based carrier, e.g., a lipid nanoparticle (LNP).

A DNA molecule or an mRNA molecule described herein may be sequenced to confirm the desired, designed sequence. In embodiments, other structural analysis of the DNA molecule or the mRNA molecule (e.g., restriction enzyme analysis) may be performed to confirm or verify its sequence.

Enrichment A composition described herein is typically enriched to remove process impurities and/or contaminants. In some embodiments, a composition comprising a DNA molecule or an mRNA molecule described herein is enriched. For instance, in some embodiments, at least 60%, at least 65%, at least 70%, at least 75%, or at least 80% by mass of total DNA in the composition may be the DNA molecule. In some embodiments, at least 60%, at least 65%, at least 70%, at least 75%, or at least 80% by mass of total RNA in the composition may be the mRNA molecule. As an example, the composition may also comprise other forms of DNA or mRNA, e.g., as a process impurity, for instance host cell DNA or host cell mRNA. As an example, the composition may comprise a contaminant, such as bacterial or viral or fungal agents.

In some embodiments, a composition described herein (e.g., a composition comprising a DNA molecule or mRNA molecule, e.g., a pharmaceutical composition comprising a DNA molecule or mRNA molecule, or a manufacturing intermediate) is free of or is substantially free of one or more process impurity or contaminant, e.g., as described in this section. In some embodiments, a method described herein results in a composition that is free of or is substantially free of one or more process impurity or contaminant, e.g., as described in this section. In some embodiments, a method described herein comprises a step of assaying for one or more process impurity or contaminant, e.g., as described in this section. In some embodiments, the method comprises approving or releasing a batch if the batch is free of or substantially free of the process impurity or contaminant or meets a release criterion for that process impurity or contaminant.

In some embodiments, the process impurity comprises a nonhuman animal serum (e.g., fetal bovine serum); an enzyme, e.g., a ligase, a polymerase, or a digestive enzyme (e.g., a trypsin, a collagenase, a DNase, a RNase, an exonuclease, or an endonuclease, e.g., a restriction endonuclease); a growth factor; a cytokine; an antibody (e.g., a monoclonal antibody); a bead (e.g., an antibody-coated bead); an antibiotic; a cell culture medium; a component of a cell culture medium; a detergent; a protein, e.g., a host cell protein; an extraneous nucleic acid sequence (e.g., a mononucleotide (e.g., a modified mononucleotide), or a DNA fragment or truncation); helper virus contaminant (e.g., infectious virus, viral DNA, or viral proteins); a solvent; a cellular debris; a cell; a pyrogen; a fungus; or any combination thereof, or a portion of any of the foregoing. In some embodiments, the contaminant was a component introduced during a manufacturing process. In some embodiments, the contaminant comprises a viral protein.

In some embodiments, the contaminant comprises an agent for transmissible spongiform encephalopathy (TSE). In some embodiments, a test for this contaminant is performed on a composition for which a bovine material was used in manufacturing.

In some embodiments, the contaminant comprises a zoonotic virus, a porcine circovirus 1, a porcine circovirus 2, or a porcine parvovirus; or any combination thereof, or a portion of any of the foregoing. In some embodiments, a test for this contaminant is performed on a composition for which non-human animal material, e.g., a porcine material, was used in manufacturing.

In some embodiments, the contaminant comprises a virus or portion thereof, e.g., a human virus; human immunodeficiency virus (HIV); HIV-1; HIV-2; hepatitis B virus (HBV); hepatitis C virus (HCV); human TSE, including Creutzfeldt-Jakob disease (CJD); variant CID (vCJD); Treponema pallidum (syphilis); human T-lymphotropic virus (HTLV), HTLV-1, HTLV-2; or cytomegalovirus, human herpesvirus (e.g., human herpesvirus-6, -7 or -8 (HHV-6, -7, or -8)), JC virus, BK virus, Epstein-Barr virus (EBV), human parvovirus B19, human papillomavirus (HPV); an adenovirus, e.g., adenovirus E1; SV40 Large T antigen sequence; HPV E6 or E7 DNA or RNA; or any combination thereof, or a portion of any of the foregoing. In some embodiments, a test for this contaminant is performed on a composition for which human donor cells (e.g., leukocyte-rich cells) were used in manufacturing. In some embodiments, a test for this contaminant is performed on a cell bank.

In some embodiments, the contaminant comprises a microbe or a portion thereof, a bacterium (e.g., a Gram-negative bacterium); mycoplasma; spiroplasma (e.g., when insect cells are used); bacterial toxin (e.g., endotoxin); or an adventitious agent, e.g., an adventitious viral agent or a non-viral adventitious agent, or any combination thereof, or a portion of any of the foregoing. In some embodiments, the contaminant comprises a simian virus, e.g., simian polyomavirus SV40 or simian retrovirus, or any combination thereof, or a portion of any of the foregoing. In some embodiments, the contaminant comprises an arbovirus. In some embodiments, the contaminant comprises a bacteriophage. In some embodiments, a test for this contaminant is performed on a cell bank, e.g., a cell bank of bacterial cells.

In some embodiments, the contaminant or process impurity comprises a DNA or mRNA from a host cell, e.g., wherein the host cell is a non-tumorigenic cell. In some embodiments, the DNA or mRNA is present at a level of less than 10 ng/dose. In some embodiments, the DNA or mRNA size is below about 200 nucleotides in length.

In some embodiments, the contaminant is an endotoxin. In some embodiments, a level of the endotoxin is less than 5 Endotoxin Unit (EU)/kg body weight/hour, e.g., wherein the composition is formulated for parenteral administration. In some embodiments, a level of the endotoxin is less than 0.2 EU/kg body weight/hour, e.g., wherein the composition is formulated for intrathecal administration. In some embodiments, a level of the endotoxin is not more than 2.0 EU/dose/eye, e.g., wherein the composition is formulated for injection or implantation into the eye, or not more than 0.5 EU/mL, e.g., wherein the composition is formulated for intraocular administration.

In some embodiments, a process impurity comprises an organic solvent, e.g., an aromatic organic solvent, e.g., phenol or chloroform.

In some embodiments, the contaminant or process impurity is described in Chemistry, Manufacturing, and Control (CMC) Information for Human Gene Therapy Investigational New Drug Applications (INDs)—Guidance for Industry (U.S. Department of Health and Human Services, Food and Drug Administration, Center for Biologics Evaluation and Research, January 2020), which is herein incorporated by reference in its entirety.

In some embodiments, the composition is substantially free of (e.g., is free of) a polymerase.

In some embodiments, the composition is substantially free of (e.g., is free of) agarose. In some embodiments, the composition is substantially free of (e.g., is free of) acrylamide.

In some embodiments, the composition is substantially free of (e.g., is free of) polypeptides.

### Pharmaceutical Compositions

The present disclosure includes a composition comprising a DNA molecule and an mRNA molecule and related compositions in combination with one or more pharmaceutically acceptable excipients and/or carriers.

Pharmaceutical compositions may optionally comprise one or more additional active substances, e.g., therapeutically and/or prophylactically active substances. Pharmaceutical compositions desctibed herein are generally sterile and/or pyrogen-free.

A DNA molecule or mRNA molecule described herein may be formulated without a carrier, e.g., the DNA molecule or mRNA molecule described herein may be administered to a host cell, tissue or subject “naked”. A naked formulation may include pharmaceutical excipients or diluents but lacks a carrier.

Pharmaceutically acceptable excipients or diluents may comprise an inactive substance that serves as a vehicle or medium for the compositions described herein, such as any one of the inactive ingredients approved by the United States Food and Drug Administration (FDA) and listed in the Inactive Ingredient Database, which is incorporated by reference herein. Non-limiting examples of pharmaceutically acceptable excipients or diluents include solvents, aqueous solvents, non-aqueous solvents, tonicity agents, dispersion media, cryoprotectants, diluents, suspension aids, surface active agents, isotonic agents, thickening agents, emulsifying agents, preservatives, hyaluronidases, dispersing agents, preservatives, lubricants, granulating agents, disintegrating agents, binding agents, antioxidants, buffering agents (e.g., phosphate buffered saline (PBS)), lubricating agents, oils, and mixtures thereof.

General considerations in the formulation and/or manufacture of pharmaceutical agents may be found, for example, in Remington: The Science and Practice of Pharmacy 21st ed., Lippincott Williams & Wilkins, 2005 (incorporated herein by reference).

In some aspects, a composition described herein (e.g., a pharmaceutical composition, e.g., a vaccine) comprises a DNA molecule described herein, an mRNA molecule described herein, and an excipient, carrier, and/or adjuvant.

In some embodiments, the vaccine described herein may further comprises one or more adjuvants. Without wishing to be bound by theories, adjuvants are agents that enhance immune responses, and their use is known in the art (see, e.g., “Vaccine Design: The Subunit and Adjuvant Approach”, Pharmaceutical Biotechnology, Volume 6, Eds. Powell and Newman, Plenum Press, New York and London, 1995). Non-limiting examples of adjuvants are complete Freund's adjuvant (CFA), incomplete Freund's adjuvant (IFA), squalene, squalane, aluminum hydroxide, aluminum salts, calcium salts, and saponin fractions derived from the bark of the South American tree Quillaja Saponaria Molina (e.g., QS21). In some embodiments the adjuvant may be an emulsion comprising oil and water. The oil phase may comprise squalene, squalane, and/or a surfactant. The surfactant may be a non-ionic surfactant, e.g., a mono- or di-Ci2-C24-fatty acid ester of sorbitan or mannide.

Synthetic variants of molecules recognized by Toll-Like Receptors (TLRs) may also be used as adjuvants. Without wishing to be bound by theory, TLRs help the body to distinguish between self and non-self molecules by recognizing molecular patterns associated with pathogens. Molecules recognized by TLRs include double-stranded RNA, lipopolysaccharides, single-stranded RNA with viral-specific or bacterial-specific modifications, and DNA with viral-specific or bacterial-specific modifications. Synthetic molecules that mimic the properties of these naturally-occurring molecules recognized by TLRs help to trigger an immune response and therefore can be used as adjuvants. Non-limiting examples of such synthetic molecules include polyriboinosinic:polyribocytidylic aci (poly (I:C)), double-stranded nucleic acids with at least one locked nucleic acid nucleoside, attenuated lipid A derivatives (ALDs) (e.g., monophosphoryl lipid A and 3-deacyl monophosphoryl lipid A), and imiquimod.

In some embodiments, a vaccine described herein may be formulated with or administered in combination with the administration of immune stimulators. Without wishing to be bound by theory, immune stimulators are molecules that increase the response of the immune system. Non-limiting examples of immune stimulators are cytokines, lymphokines, and chemokines that have immunostimulatory, immunopotentiating, and pro-inflammatory activities, such as interleukins (e.g., IL-1, IL-2, IL-3, IL-4, IL-12, IL-13), growth factors (e.g., granulocyte-macrophage (GM)-colony stimulating factor (CSF)); and other immunostimulatory molecules, such as macrophage inflammatory factor, Flt3 ligand, B7.1; B7.2, etc. Immune stimulators may be administered in the same formulation as a composition described herein, e.g., a composition comprising a DNA molecule or mRNA molecule described herein, or may be administered separately from the composition. Immune stimulators may be administered as proteins or as nucleic acids from which the immunostimulatory protein can be expressed.

Carriers A DNA molecule or an mRNA molecule described herein may also be formulated, or included, with a carrier. General considerations of carriers and delivery of pharmaceutical agents may be found, for example, in “Delivery Technologies for Biopharmaceuticals: Peptides, Proteins, Nucleic Acids and Vaccines” (Lene Jorgensen and Hanne Morck Nielson, Eds.) Wiley; 1st edition (Dec. 21, 2009); and Vargason et al. 2021. Nat Biomed Eng 5, 951-967. Non-limiting examples of carriers include carbohydrate carriers (e.g., an anhydride-modified phytoglycogen or glycogen-type material, GalNAc), nanoparticles (e.g., a nanoparticle that encapsulates or is covalently linked to the DNA molecule or the mRNA molecule, gold nanoparticles, silica nanoparticles), lipid particles (e.g., liposomes, lipid nanoparticles), cationic carriers (e.g., a cationic lipopolymer or transfection reagent), fusosomes, non-nucleated cells (e.g., ex vivo differentiated reticulocytes), nucleated cells, exosomes, protein carriers (e.g., a protein covalently linked to the DNA molecule or the mRNA molecule), peptides (e.g., cell-penetrating peptides), materials (e.g., graphene oxide), single pure lipids (e.g., cholesterol), DNA origami (e.g., DNA tetrahedron).

In one embodiment, the DNA molecules or the mRNA molecules, compositions, constructs and systems described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Exosomes can also be used as drug delivery vehicles for the compositions and systems described herein. For a review, see Ha et al. July 2016. Acta Pharmaceutica Sinica B. Volume 6, Issue 4, Pages 287-296; doi.org/10.1016/j.apsb.2016.02.001.

Ex vivo differentiated red blood cells can also be used as a carrier for an agent (e.g., a DNA molecule or an mRNA molecule) described herein. See, e.g., WO2015073587; WO2017123646; WO2017123644; WO2018102740; WO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014.

### Proc Natl Acad Sci USA. 111(28): 10131-10136.

Fusosome compositions, e.g., as described in WO2018208728, can also be used as carriers to deliver the DNA molecules or the mRNA molecules described herein.

### Lipid Nanoformulations Lipid-Based Carriers

In some embodiments, compounds, e.g., DNA molecules or mRNA molecules, described herein are formulated into a lipid-based carrier (or lipid nanoformulation). In some embodiments, the lipid-based carrier (or lipid nanoformulation) is a liposome or a lipid nanoparticle (LNP). In one embodiment, the lipid-based carrier is an LNP.

In some embodiments, the lipid-based carrier (or lipid nanoformulation) comprises a cationic lipid (e.g., an ionizable lipid), a non-cationic lipid (e.g., phospholipid), a structural lipid (e.g., cholesterol), and a PEG-modified lipid. In some embodiments, the lipid-based carrier (or lipid nanoformulation) contains one or more compounds described herein, or a pharmaceutically acceptable salt thereof.

As described herein, suitable compounds to be used in the lipid-based carrier (or lipid nanoformulation) include all the isomers and isotopes of the compounds described above, as well as all the pharmaceutically acceptable salts, solvates, or hydrates thereof, and all crystal forms, crystal form mixtures, and anhydrides or hydrates.

In addition to one or more compounds described herein, the lipid-based carrier (or lipid nanoformulation) may further include a second lipid. In some embodiments, the second lipid is a cationic lipid, a non-cationic (e.g., neutral, anionic, or zwitterionic) lipid, or an ionizable lipid.

One or more naturally occurring and/or synthetic lipid compounds may be used in the preparation of the lipid-based carrier (or lipid nanoformulation).

The lipid-based carrier (or lipid nanoformulation) may contain positively charged (cationic) lipids, neutral lipids, negatively charged (anionic) lipids, or a combination thereof.

### Cationic Lipids (Positively Charged) and Ionizable Lipids

In some embodiments, the lipid-based carrier (or lipid nanoformulation) comprises one or more cationic lipids, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N′-dimethyl-N,N′-dioctacyl ammonium bromide (DDAB) and chloride DDAC), N-(1-(2,3-dioleyloxy)propyl)-N,N,N-trimethylammonium chloride (DOTMA), 3β-[N—(N′,N′-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), N,N-dioleyl-N,N-dimethylammonium chloride (DODAC), N,N-dimethyl-2,3-dioleyloxy)propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), 3-Dimethylamino-2-(Cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis, cis-9′,12′-octadecadienoxy)propane (CpLin DMA), N,N-Dimethyl-3,4-dioleyloxybenzylamine (DMOBA), and the cationic lipids described in e.g. Martin et al., Current PharmaceuticalDesign, pages 1-394, which is herein incorporated by reference in its entirety. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, which is incorporated by reference.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein. Exemplary ionizable lipids include, but are not limited to,

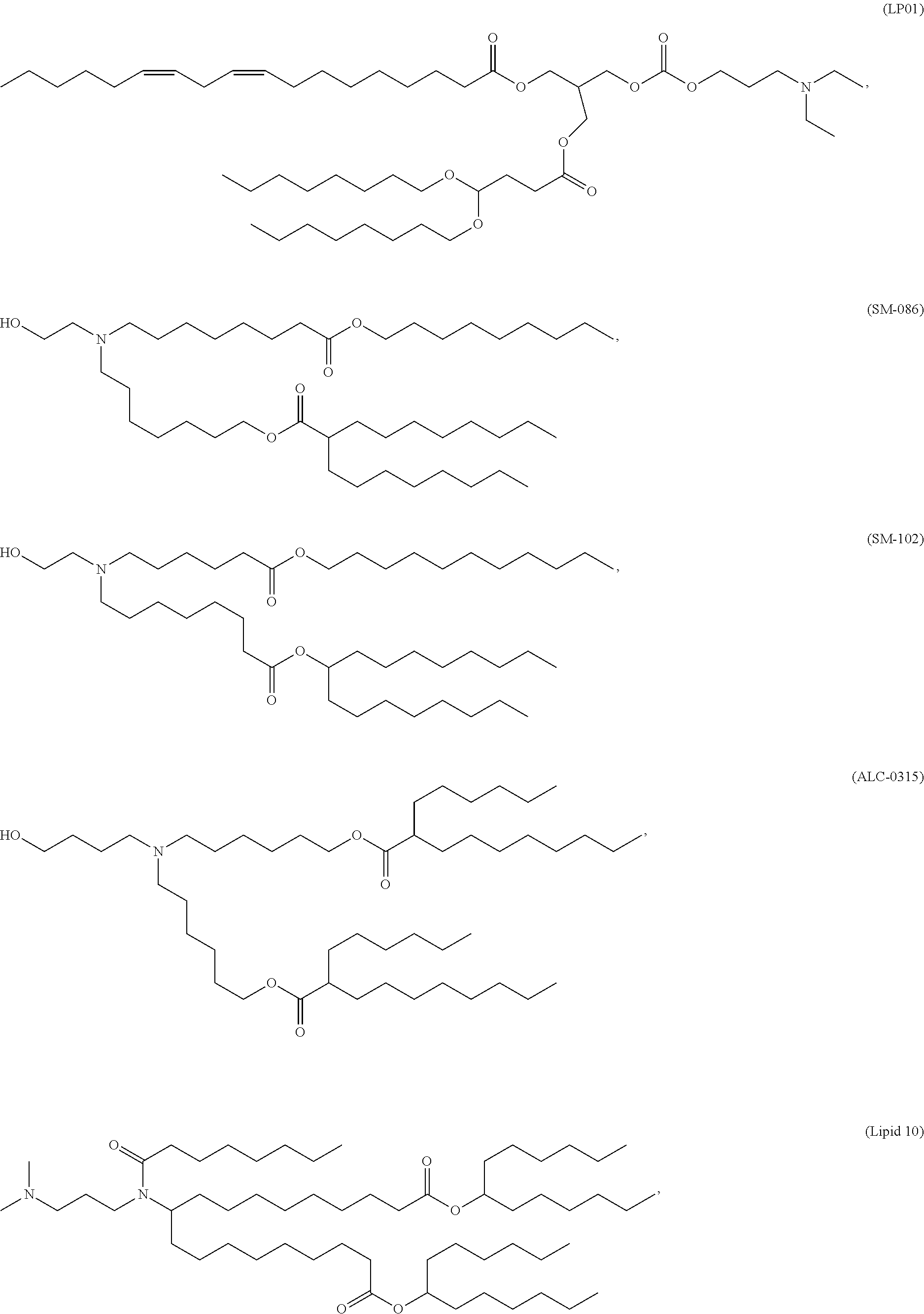

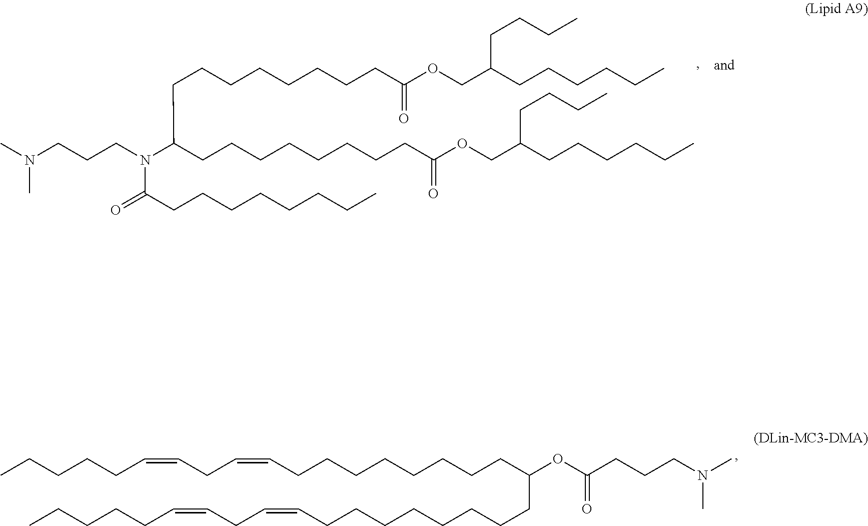

(see WO 2017/004143A1, which is incorporated herein by reference in its entirety).

In some embodiments, the lipid-based carrier (or lipid nanoformulation) further comprises one or more compounds described by WO 2021/113777 (e.g., a lipid of Formula (3) such as a lipid of Table 3 of WO 2021/113777), which is incorporated herein by reference in its entirety.

In one embodiment, the ionizable lipid is a lipid disclosed in Hou, X., et al. Nat Rev Mater 6, 1078-1094 (2021). doi.org/10.1038/s41578-021-00358-0 (e.g., L319, C12-200, and DLin-MC3-DMA), (which is incorporated by reference herein in its entirety).

Examples of other ionizable lipids that can be used in lipid-based carrier (or lipid nanoformulation) include, without limitation, one or more of the following formulas: X of US 2016/0311759; I of US 20150376115 or in US 2016/0376224; Compound 5 or Compound 6 in US 2016/0376224; I, IA, or II of U.S. Pat. No. 9,867,888; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y-Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; 111-3 of WO 2018/081480; I-5 or I-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7C1 of Dahlman et al (2017); 304-013 or 503-013 of Whitehead et al; TS-P4C2 of U.S. Pat. No. 9,708,628; I of WO 2020/106946; I of WO 2020/106946; (1), (2), (3), or (4) of WO 2021/113777; and any one of Tables 1-16 of WO 2021/113777, all of which are incorporated herein by reference in their entirety.

In some embodiments, the lipid-based carrier (or lipid nanoformulation) further includes biodegradable ionizable lipids, for instance, (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, which are incorporated herein by reference in their entirety.

Non-Cationic Lipids (e.g., Phospholipids) In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl- phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl- sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, which is incorporated herein by reference. Such lipids include, in some embodiments, plant lipids found to improve liver transfection with mRNA (e.g., DGTS).

In some embodiments, the lipid-based carrier (or lipid nanoformulation) may comprise a combination of distearoylphosphatidylcholine/cholesterol, dipalmitoylphosphatidylcholine/cholesterol, dimyrystoylphosphatidylcholine/cholesterol, 1,2-Dioleoyl-sn-glycero-3-phosphocholine (DOPC)/cholesterol, or egg sphingomyelin/cholesterol. Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO 2017/099823 or US 2018/0028664, which are incorporated herein by reference in their entirety.

In one embodiment, the lipid-based carrier (or lipid nanoformulation) further comprises one or more non-cationic lipid that is oleic acid or a compound of Formula I, II, or IV of US 2018/0028664, which is incorporated herein by reference in its entirety.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

### Structural Lipids

The lipid-based carrier (or lipid nanoformulation) described herein may further comprise one or more structural lipids. As used herein, the term “structural lipid” refers to sterols (e.g., cholesterol) and also to lipids containing sterol moieties.

Incorporation of structural lipids in the lipid nanoparticle may help mitigate aggregation of other lipids in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO 2009/127060 or US 2010/0130588, which are incorporated herein by reference in their entirety. Additional exemplary sterols include phytosterols, including those described in Eygeris et al. (2020), Nano Lett. 2020; 20(6):4543-4549, incorporated herein by reference.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in WO 2009/127060 and US 2010/0130588, each of which is incorporated herein by reference in its entirety.

In some embodiments, the lipid-based carrier (or lipid nanoformulation) further comprises sterol in an amount of 0-50 mol % (e.g., 0-10 mol %, 10-20 mol %, 20-50 mol %, 20-30 mol %, 30-40 mol %, or 40-50 mol %) of the total lipid components.

### Polymers and Polyethylene Glycol (PEG)—Lipids

In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid. Examples of useful PEG-lipids include, but are not limited to, 1,2-Diacyl-sn-Glycero-3-Phosphoethanolamine-N-[Methoxy(Polyethylene glycol)-350](mPEG 350 PE); 1,2-Diacyl-sn- Glycero-3-Phosphoethanolamine-N-[Methoxy(Polyethylene glycol)-550](mPEG 550 PE); 1,2-Diacyl-sn-Glycero-3-Phosphoethanolamine-N-[Methoxy(Polyethylene glycol)-750](mPEG 750 PE); 1,2-Diacyl-sn-Glycero-3-Phosphoethanolamine-N-[Methoxy(Polyethylene glycol)-1000](mPEG 1000 PE); 1,2-Diacyl-sn-Glycero-3-Phosphoethanolamine-N-[Methoxy(Polyethylene glycol)-2000](mPEG 2000 PE); 1,2-Diacyl-sn-Glycero-3-Phosphoethanolamine-N-[Methoxy(Polyethylene glycol)-3000](mPEG 3000 PE); 1,2-Diacyl-sn-Glycero-3-Phosphoethanolamine-N-[Methoxy(Polyethylene glycol)-5000](mPEG 5000 PE); N-Acyl- Sphingosine-1-[Succinyl(Methoxy Polyethylene Glycol) 750](mPEG 750 Ceramide); N-Acyl- Sphingosine-1-[Succinyl(Methoxy Polyethylene Glycol) 2000](mPEG 2000 Ceramide); and N-Acyl-Sphingosine-1-[Succinyl(Methoxy Polyethylene Glycol) 5000](mPEG 5000 Ceramide). In some embodiments, the PEG lipid is a polyethyleneglycol-diacylglycerol (i.e., polyethyleneglycol diacylglycerol (PEG-DAG), PEG-cholesterol, or PEG-DMB) conjugate.

In some embodiments, the lipid-based carrier (or nanoformulation) includes one or more conjugated lipids (such as PEG-conjugated lipids or lipids conjugated to polymers described in Table 5 of WO 2019/217941, which is incorporated herein by reference in its entirety). In some embodiments, the one or more conjugated lipids is formulated with one or more ionic lipids (e.g., non-cationic lipid such as a neutral or anionic, or zwitterionic lipid); and one or more sterols (e.g., cholesterol).

The PEG conjugate can comprise a PEG-dilaurylglycerol (C12), a PEG-dimyristylglycerol (C14), a PEG-dipalmitoylglycerol (C16), a PEG-disterylglycerol (C18), PEG-dilaurylglycamide (C12), PEG-dimyristylglycamide (C14), PEG-dipalmitoylglycamide (C16), and PEG-disterylglycamide (C18).

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG- ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w- methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (which is herein incorporated by reference in its entirety), and combinations of the foregoing.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, all of which are incorporated herein by reference in their entirety.

In some embodiments, the PEG-lipid is a compound of Formula III, III-a-I, III-a-2, III-b-1, III-b-2, or V of US 2018/0028664, which is incorporated herein by reference in its entirety. In some embodiments, the PEG-lipid is of Formula II of US 2015/0376115 or US 2016/0376224, both of which are incorporated herein by reference in their entirety. In some embodiments, the PEG-DAA conjugate can be, for example, PEG-dilauryloxypropyl, PEG- dimyristyloxypropyl, PEG-dipalmityloxypropyl, or PEG-distearyloxypropyl. In some embodiments, the PEG-lipid includes one of the following:

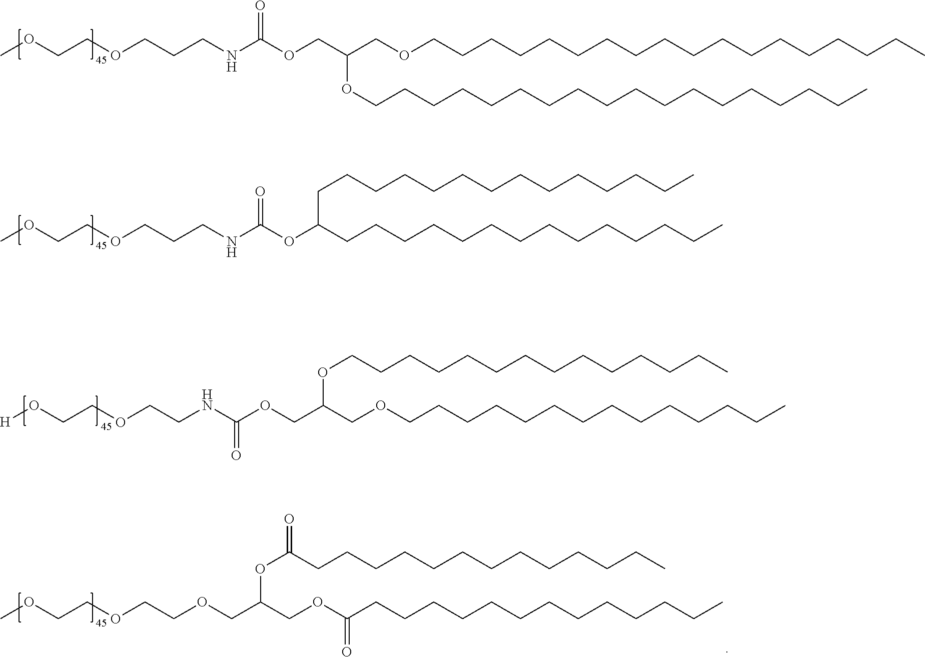

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, which is incorporated herein by reference in its entirety.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol % or 2-5 mol % of the total lipid components.

When needed, the lipid-based carrier (or lipid nanoformulation) described herein may be coated with a polymer layer to enhance stability in vivo (e.g., sterically stabilized LNPs).

Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther, 289: 1128-1133 (1999); Gabizon et al., JControlled Release 53: 275-279 (1998); Adlakha Hutcheon et al., Nat Biotechnol 17: 775-779 (1999); and Koning et al., Biochim Biophys Acta 1420: 153-167 (1999), which are incorporated herein by reference in their entirety.

### Percentages of Lipid Nanoformulation Components

In some embodiments, the lipid-based carrier (or lipid nanoformulation) comprises one of more of the compounds described herein, optionally a non-cationic lipid (e.g., a phospholipid), a sterol, a neutral lipid, and optionally conjugated lipid (e.g., a PEGylated lipid) that inhibits aggregation of particles. In some embodiments, the lipid-based carrier (or lipid nanoformulation) further comprises a payload (e.g., a DNA molecule or an mRNA molecule described herein). The amounts of these components can be varied independently and to achieve desired properties. For example, in some embodiments, the ionizable lipid including the lipid compounds described herein is present in an amount from about 20 mol % to about 100 mol % (e.g., 20-90 mol %, 20-80 mol %, 20-70 mol %, 25-100 mol %, 30-70 mol %, 30-60 mol %, 30-40 mol %, 40-50 mol %, or 50-90 mol %) of the total lipid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid components, a conjugated lipid (e.g., a PEGylated lipid) in an amount from about 0.5 mol % to about 20 mol % (e.g., 1-10 mol % or 5-10%) of the total lipid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-40 mol %) of the total lipid components, provided that the total mol % of the lipid component does not exceed 100%.

In some embodiments, the lipid-based carrier (or lipid nanoformulation) comprises about 25-100 mol % of the ionizable lipid including the lipid compounds described herein, about 0-50 mol % phospholipid, about 0-50 mol % sterol, and about 0-10 mol % PEGylated lipid.

In some embodiments, the lipid-based carrier comprises a payload (e.g., a DNA molecule or an mRNA molecule described herein, etc.) that is formulated in a lipid nanoparticle, wherein the lipid nanoparticle comprises about 25-100 mol % of the ionizable lipid including the lipid compounds described herein, about 0-50 mol % phospholipid, about 0-50 mol % sterol, and about 0-10 mol % PEGylated lipid. In some embodiments, the encapsulation efficiency of the payload may be at least 70%.

In one embodiment, the lipid-based carrier (or lipid nanoformulation) comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier comprises a payload (e.g., a DNA molecule or an mRNA molecule described herein) that is formulated in a lipid nanoparticle, wherein the lipid nanoparticle comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid. In some embodiments, the encapsulation efficiency of the payload may be at least 70%.

In some embodiments, the lipid-based carrier (or lipid nanoformulation) comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, the lipid-based carrier comprises a payload (e.g., a DNA molecule or an mRNA molecule described herein) that is formulated in a lipid nanoparticle, wherein the lipid nanoparticle comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid. In some embodiments, the encapsulation efficiency of the payload may be at least 70%.

In some embodiments, molar ratios of ionizable lipid/sterol/phospholipid (or another structural lipid)/PEG-lipid/additional components is varied in the following ranges: ionizable lipid (25-100%); phospholipid (DSPC) (0-40%); sterol (0-50%); and PEG lipid (0-5%).

In some embodiments, the lipid-based carrier comprises a payload (e.g., a DNA molecule or an mRNA molecule described herein) that is formulated in a lipid nanoparticle, wherein the lipid nanoparticle comprises molar ratios of ionizable lipid/sterol/phospholipid (or another structural lipid)/PEG-lipid/additional components in the following ranges: ionizable lipid (25-100%); phospholipid (DSPC) (0-40%); sterol (0-50%); and PEG lipid (0-5%). In some embodiments, the encapsulation efficiency of the payload may be at least 70%.

In some embodiments, the lipid-based carrier (or lipid nanoformulation) comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid).

In some embodiments, the lipid-based carrier comprises a payload (e.g., a DNA molecule or an mRNA molecule described in) that is formulated in a lipid nanoparticle, wherein the lipid nanoparticle comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid). In some embodiments, the encapsulation efficiency of the payload may be at least 70%.

In some embodiments, the lipid-based carrier (or lipid nanoformulation) comprises (i) a DNA molecule or an mRNA molecule; (ii) a cationic lipid comprising from 50 mol % to 65 mol % of the total lipid present in the lipid-based carrier; (iii) a non-cationic lipid comprising a mixture of a phospholipid and a cholesterol derivative thereof, wherein the phospholipid comprises from 3 mol % to 15 mol % of the total lipid present in the lipid-based carrier and the cholesterol or derivative thereof comprises from 30 mol % to 40 mol % of the total lipid present in the lipid-based carrier; and (iv) a conjugated lipid comprising 0.5 mol % to 2 mol % of the total lipid present in the particle.

In some embodiments, the lipid-based carrier (or lipid nanoformulation) comprises (i) a DNA molecule or an mRNA molecule; (ii) a cationic lipid comprising from 50 mol % to 85 mol % of the total lipid present in the lipid-based carrier; (iii) a non-cationic lipid comprising from 13 mol % to 49.5 mol % of the total lipid present in the lipid-based carrier; and (d) a conjugated lipid comprising from 0.5 mol % to 2 mol % of the total lipid present in the lipid-based carrier.

In some embodiments, the phospholipid component in the mixture may be present from 2 mol % to 20 mol %, from 2 mol % to 15 mol %, from 2 mol % to 12 mol %, from 4 mol % to 15 mol %, from 4 mol % to 10 mol %, from 5 mol % to 10 mol %, (or any fraction of these ranges) of the total lipid components. In some embodiments, the lipid-based carrier (or lipid nanoformulation) is phospholipid-free.

In some embodiments, the sterol component (e.g. cholesterol or derivative) in the mixture may comprise from 25 mol % to 45 mol %, from 25 mol % to 40 mol %, from 25 mol % to 35 mol %, from 25 mol % to 30 mol %, from 30 mol % to 45 mol %, from 30 mol % to 40 mol %, from 30 mol % to 35 mol %, from 35 mol % to 40 mol %, from 27 mol % to 37 mol %, or from 27 mol % to 35 mol % (or any fraction of these ranges) of the total lipid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid components.

The ratio of total lipid components to the payload (e.g., an encapsulated therapeutic agent such as a DNA molecule or an mRNA molecule) can be varied as desired. For example, the total lipid components to the payload (mass or weight) ratio can be from about 10:1 to about 30:1. In some embodiments, the total lipid components to the payload ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the payload can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulation's) overall lipid content can range from about 5 mg/ml to about 30 mg/mL. Nitrogen:phosphate ratios (N:P ratio) is evaluated at values between 0.1 and 100.

The efficiency of encapsulation of a payload such as a DNA molecule or an mRNA molecule, describes the amount of the DNA molecule or the mRNA molecule that is encapsulated or otherwise associated with a lipid nanoformulation (e.g., liposome or LNP) after preparation, relative to the initial amount provided. The encapsulation efficiency is desirably high (e.g., at least 70%, 80%, 90%, 95%, or close to 100%). The encapsulation efficiency may be measured, for example, by comparing the amount of DNA molecule or mRNA molecule in a solution containing the liposome or LNP before and after breaking up the liposome or LNP with one or more organic solvents or detergents. An anion exchange resin may be used to measure the amount of DNA molecule or mRNA molecule in a solution. Fluorescence may be used to measure the amount of DNA molecule or mRNA molecule in a solution. For the lipid-based carrier (or lipid nanoformulation) described herein, the encapsulation efficiency of a DNA molecule or an mRNA molecule may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 70%. In some embodiments, the encapsulation efficiency may be at least 80%. In some embodiments, the encapsulation efficiency may be at least 90%. In some embodiments, the encapsulation efficiency may be at least 95%.

In some embodiments, the disclosure provides a delivery device comprising the mRNA molecule and the DNA molecule. In some embodiments, the disclosure provides a first delivery device comprising the mRNA molecule and a second delivery device comprising the DNA molecule. In some embodiments, the delivery device is a syringe.

Route of administration A DNA molecule or an mRNA molecule described herein may be introduced into a cell, tissue or subject by any suitable route.

Administration to a target cell or tissue (e.g., ex vivo) may be by methods known in the art such as transfection, e.g., transient or stable transfection using reagents (e.g., liposomal, calcium phosphate) or physical means (e.g., electroporation, gene gun, microinjection, microfluidic fluid shear, cell squeezing). Other methods are described, e.g., in Rad et al. 2021. Adv. Mater. 33:2005363, which is incorporated herein by reference.

Administration to a subject, e.g., a mammal, e.g., a human subject, may be by parenteral (e.g., intravenous, intramuscular, intraperitoneal, subcutaneous, or intracranial) route; by topical administration, transdermal administration or transcutaneous administration. For instance, in some embodiments, the composition is a vaccine and is administrated by the intramuscular route, subcutaneous route, intranasal route, or oral route. In some embodiments, the composition is a vaccine and is administered by the intramuscular route, e.g., via intramuscular administration to a quadricep muscle. Other suitable routes include oral, rectal, transmucosal, intranasal, inhalation (e.g., via an aerosol), buccal (e.g., sublingual), vaginal, intrathecal, intraocular, transdermal, intraendothelial, in utero (or in ovo), intrapleural, intracerebral, intraarticular, topical, intralymphatic. Also included is direct tissue or organ injection (e.g., to liver, eye, skeletal muscle, cardiac muscle, diaphragm, muscle or brain).

### Kits

The present disclosure provides, for instance, kits comprising a first container comprising a mRNA molecule and a second container comprising a DNA molecule as described herein. In some embodiments, the kit is for therapeutic use. In some embodiments, the kit further comprises instructions for therapeutic use. In some embodiments, the kit is for resesarch use. In some embodiments, the kit further comprises instructions for research use. In some embodiments, the kit further comprises an additional reagent. In some embodiments, the kit further comprises a delivery device such as a syringe.

### Applications

A composition (e.g., pharmaceutical composition, e.g., pharmaceutical composition comprising a DNA molecule and an mRNA molecule) described herein can be used in therapeutic or health applications for a subject, e.g., a human or non-human animal. Although the descriptions of pharmaceutical compositions provided herein are principally directed to pharmaceutical compositions which are suitable for administration to humans, it will be understood by the skilled artisan that such compositions are generally suitable for administration to any other animal. The subject can be any animal, e.g., a mammal, e.g., a human or non-human mammal. In embodiments, the subject is a vertebrate animal (e.g., mammal, bird, fish, reptile, or amphibian). In embodiments, the subject is a human. In embodiments, the method subject is a non-human mammal. In embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusk.

In some embodiments, a DNA molecule or an mRNA molecule described herein is provided at a dose of about 0.1-100 mg/kg of DNA or mRNA.

In some embodiments, a system described herein (e.g., comprising a nucleic acid molecule, e.g., comprising a dsDNA molecule and/or an RNA molecule) is provided at a dose of g to 300 g nucleic acid molecules, e.g., when administered via intramuscular route. In some embodiments, a system described herein (e.g., comprising a nucleic acid molecule, e.g., comprising a dsDNA molecule and/or an RNA molecule) is provided at a dose of 0.001 mg/kg to 2 mg/kg nucleic acid molecules, e.g., when administered via intravenous route.

In some embodiments, a DNA molecule or an mRNA molecule described herein imparts a biological effect of the effector, e.g., expression of a therapeutic polypeptide, on a host cell, tissue or subject over a time period of at least 2, at least 3, at least 4, at least 5, at least 6 days or at least a week; at least 8, at least 9, at least 10, at least 12, at least 14 days or at least two weeks; at least 16, at least 18, at least 20 days or at least 3 weeks; at least 22, at least 24, at least 25, at least 27, at least 28 days or at least a month; at least 2 months, at least 3 months, at least 4 months, at least 5 months, at least 6 months or more; between one week and 6 months, between 1 month to 6 months, or between 3 months to 6 months.

In some embodiments, a DNA molecule or an mRNA molecule described herein imparts a biological effect of the effector, e.g., expression of a therapeutic polypeptide, on a host cell, tissue or subject over a time period of at least 1 cell divisions of the host cell.

In embodiments, a DNA molecule or an mRNA molecule described herein can be used to deliver an effector, e.g., an effector described herein, to a cell, tissue or subject.

In embodiments, a DNA molecule or an mRNA molecule described herein can be used to modulate (e.g., increase or decrease) a biological parameter in a cell, tissue or subject. The biological parameter may be an increase or decrease in gene expression of a subject gene in a target cell, tissue or subject. In some embodiments, a DNA molecule or an mRNA molecule described herein increases or decreases a biological activity in a target cell, wherein the biological activity comprises cell growth, cell metabolism, cell signaling, cell movement, specialization, interactions, division, transport, homeostasis, osmosis, or diffusion. In some embodiments, the cell is an animal cell, e.g., a mammalian cell, e.g., a human cell.

In embodiments, a DNA or an mRNA molecule molecule described herein can be used to treat a cell, tissue or subject in need thereof by administering the DNA molecule or an mRNA molecule described herein to such cell, tissue or subject.

In embodiments, the DNA molecule or an mRNA molecule delivers an effector to a cell.

In some aspects, a composition described herein comprises an immunogenic composition, and an excipient, carrier, and/or adjuvant, wherein the composition is for nasal administration. In some aspects, the composition comprises an immunogenic composition, and an excipient, carrier, and/or adjuvant, wherein the composition is for subcutaneous administration, and the adjuvant is an aluminum-based adjuvant, preferably alum. In some aspects, the composition comprises an immunogenic composition, and an excipient, carrier, and/or adjuvant, wherein the composition is for intramuscular administration, and the adjuvant is an aluminum-based adjuvant, for instance alum. In some aspects, the composition is administered to the subject more than once, for instance at least two times to the subject, with between 2-6 weeks in between each administration. In some embodiments, the subject is a male or female. In some embodiments, the subject is an adult or a pediatric subject.

### EXAMPLES

### Example 1: Production of Lipid Nanoparticles (LNPs) Comprising DNA Molecules or mRNA Molecules

This Example demonstrates the encapsulation of DNA molecules or mRNA molecules encoding an antigen into LNPs prior to dosing into mice.

Lipid nanoparticles comprising an ionizable lipid were formulated encapsulating either circular dsDNA (cdsDNA) molecules or linear mRNA molecules encoding a Covid Spike peptide using standard formulation methods. The mRNA molecules comprise 5-methoxyuracil. In brief, the mRNA molecule or DNA molecules were diluted into citrate buffer (pH 4.0), and the lipid mix (50% ionizable lipid, 10% DSPC, 38.5% Cholesterol and 1.5% DMG-PEG2000 by mole) was dissolved into ethanol. The two solutions were mixed rapidly at a ratio of 3:1 (mRNA, DNA, or the combination of mRNA and DNA:lipids, vol:vol). Lipid nanoparticles were washed and concentrated using Amicon centrifugal filter units (100 kDa, UFC8100, Millipore) before characterization.

### Example 2: In Vivo Study to Assess mRNA DNA Vaccine Combinations

This Example demonstrates the efficacy of the mRNA vaccine, DNA vaccine, and mRNA/DNA combination vaccine in a mouse model.

The mRNA vaccine, DNA vaccine, or mRNA/DNA combination vaccine, produced as described in Example 1, was prepared to a final concentration of 0.2 mg/mL in PBS and loaded into a sterile syringe. The mRNA vaccine, DNA vaccine, or mRNA/DNA combination vaccine was infused unilaterally into the gastrocnemius muscle to a total dose of 5 μg per mouse. Following administration of the vaccine, the mice were recovered from anesthesia and returned to their cages. For each group of mice receiving the mRNA vaccine, DNA vaccine, or mRNA/DNA combination vaccine respectively, a sub-group of mice then received a second administration 14 days following the first administration, wherein the second administration was identical to the first administration. All mice were monitored for clinical signs and other phenotypic indications of adverse events for the course of the study.

On day 35 or 56 after the first vaccine administration, the mice were euthanized, and 0.5-1 mL of whole blood was drawn through cardiac puncture. Secondary lymphoid organs including spleen and draining lymph nodes were collected and dissociated into single cell suspensions.

To assess antigen specific T-cell responses against Covid Spike, the dissociated cells were first incubated (37° C., 5% CO₂) in T-cell maintenance medium (in the presence of mIL2, 5 ng/mL, StemCell 78-81) and a Covid Spike Peptide array (PepTivator SARS-CoV-2, Miltenyi) for 2 hrs. Brefeldin A (00-4506-51, Invitrogen) was then added, and cells were incubated for an additional 4 hrs (37° C., 5% CO₂). Following incubations, CD4 or CD8 cell markers were immunolabeled and assessed via flow cytometry.

Serological Anti-SARS-Cov-2 Antibody was assessed from blood plasma via ELISA (Acro Biosciences, RAS-T03). In order to determine the ability of blood plasma to prevent binding of angiotensin-converting enzyme 2 (ACE2) to Spike coated beads (Biolegend 741328), a custom dilution-based competition assay was used to assess neutralization antibody titer. In brief, the plasma samples were diluted in sample diluent buffer (PBS+2% BSA) to assay dilutions. Spike coated beads (Biolegend 741328) were diluted to 1000 beads/50 uL with assay buffer (Biolegend 77562). Diluted plasma analytes were added to bead solution. Phycoerythrin (PE)-conjugated ACE2 was added to the mixed solution with a final concentration of 0.33 ug/uL. After incubation, the beads were washed in wash buffer and assessed via flow cytometry.

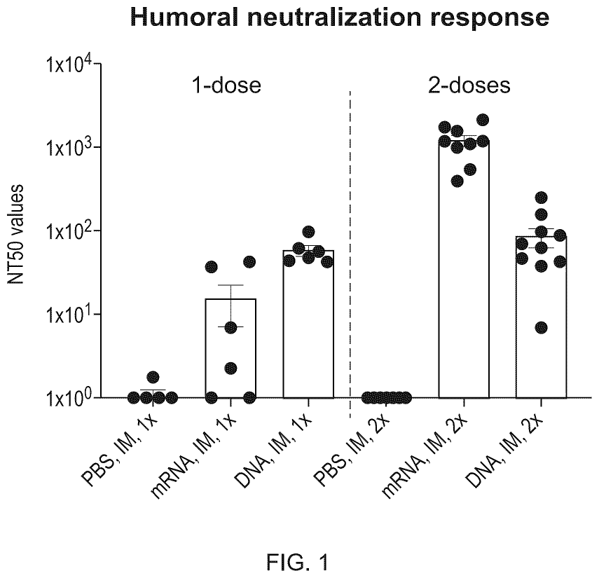

As shown in FIG. 1, the humoral neutralization response, represented by the blood plasma 50% neutralizing titer 35 days after the first dose, was detected in mice receiving either the mRNA vaccine or the DNA vaccine, for one or two doses.

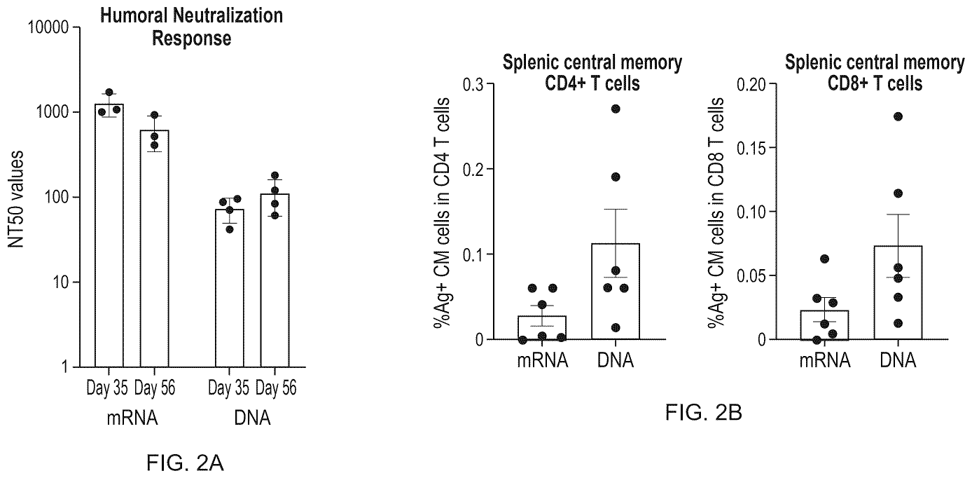

FIG. 2A shows that the humoral neutralization response in mice receiving mRNA vaccine is detectable at day 35 and day 56 following the first administration. FIG. 2B shows CD4 or CD8 T cells specifically recognized Covid Spike isolated from dissociated spleen of mice receiving the DNA vaccine or the mRNA vaccine.

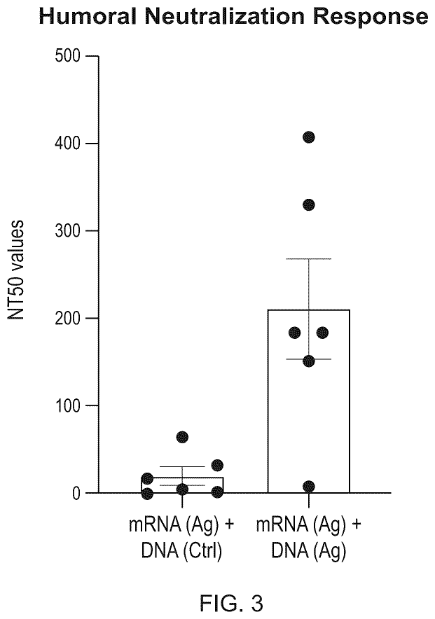

FIG. 3 shows that the humoral neutralization response was significantly higher in mice receiving a mRNA/DNA combination vaccine compared to mice receiving mRNA vaccine only.

### Example 3: Non-Human Primate Study to Assess DNA Vaccines and mRNA Vaccines

This Example demonstrates the efficacy of DNA vaccines and mRNA vaccines in inducing cellular immunity in cynomolgus macaque non-human primates.

Lipid nanoparticles (LNPs) were formulated encapsulating circular “hemi-modified” dsDNA molecules or linear mRNA molecules encoding a Covid Spike peptide using standard formulation methods. The circular hemi-modified dsDNA molecules were produced in a reaction which included complete incorporation of canonical uracil, in place of thymine, on the sense strand. The mRNA molecules or DNA molecules were diluted into citrate buffer (pH 4.0) and the lipid mix (50% ionizable lipid, 10% DSPC, 38.5% Cholesterol and 1.5% DMG-PEG2000 by mole) was dissolved into ethanol. The two solutions were mixed rapidly at a ratio of 3:1 (mRNA or DNA:lipids, vol:vol), and the resulting LNPs were washed and concentrated using Amicon centrifugal filter units (100 kDa, UFC8100, Millipore) before characterization.

The vaccines comprising the LNPs were prepared to a final concentration of 0.01 mg/mL (mRNA) or 0.025 mg/mL (DNA). On study day 1, animals were dosed into the NHP quadriceps muscle via intramuscular administration with either the mRNA vaccine (for a total dose of 20 ug mRNA per animal) or the DNA vaccine (for a total dose of 50 ug DNA per animal). On day 22, all animals received a dose of the mRNA vaccine. Following each dose, animals were monitored for clinical signs and other phenotypic indications of adverse events for the course of the study.

On specified days after administration of the vaccines, blood was drawn and PBMCs were isolated. To assess antigen specific T-cell responses against Covid Spike, the isolated PBMCs were incubated (37° C., 5% CO₂) in RPMI 1640 containing 2 mM L-Glutamine (Gibco, Cat 11875), supplemented with 10% FBS (Gibco, Cat. 16140071), PenStrep (Gibco, Cat: 15140122), 50 uM Beta-Mercaptoethanol (Gibco, Cat 21985023) and a Covid Spike Peptide array (JPT Peptide Technologies, Cat: PM-WCPV-S-1) at 2 μg/mL for 16 hrs. Covid spike specific cellular response was assessed via ELISpot (Mabtech 3421M-4APW-10).

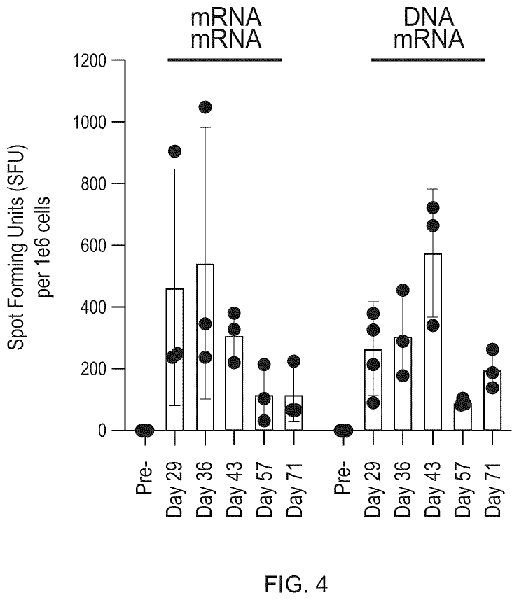

As shown in FIG. 4, PBMCs collected from the animals at various time points (day 29, 36, 43, 57 and 71 following the initial dose of the mRNA vaccine or DNA vaccine) exhibited antigen-specific cellular response, as measured by an ELIspot assay for IFN gamma (IFNg) levels following incubation of the PBMCs with a Covid Spike peptide array. The data demonstrates that the DNA and mRNA vaccines induced durable cellular immunity following administration to the subject.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., PubChem compound, PubChem substance, or PubChem Bioassay entries, including the annotations therein, such as structures and assays, et cetera), are hereby incorporated by reference in their entirety.

Headings used in this application are for convenience only and do not affect the interpretation of this application.

1. A pharmaceutical composition comprising:
(a) an mRNA molecule encoding a first immunogen; and
(b) a double stranded DNA molecule encoding: (i) the first immunogen, (ii) a second immunogen having at least 90% amino acid sequence identity to the first immunogen, or (iii) a second immunogen that is recognized by an antigen binding site of an antibody or T cell receptor (TCR), wherein the antigen binding site also recognizes the first immunogen.

2. The pharmaceutical composition of claim 1, wherein the first immunogen is a viral antigen, a bacterial antigen, a fungal antigen, or a tumor antigen.

3. The pharmaceutical composition of claim 1, wherein the mRNA molecule comprises a 5′ cap or a polyA tail.

4. (canceled)

5. The pharmaceutical composition of claim 1, wherein the DNA molecule comprises a plasmid or a minicircle.

6.-9. (canceled)

10. The pharmaceutical composition of claim 1, wherein the DNA molecule comprises a promoter sequence operably linked to the sequence encoding the first immunogen.

11. The pharmaceutical composition of claim 1, wherein the mRNA molecule and DNA molecule are present at a molar ratio of between 10:1 and 1:10 mRNA:DNA.

12. The pharmaceutical composition of claim 1, wherein the pharmaceutical composition is free of protein.

13. The pharmaceutical composition of claim 1, wherein the pharmaceutical composition is free of virus.

14. The pharmaceutical composition of claim 1, wherein the pharmaceutical composition comprises a pharmaceutically acceptable adjuvant.

15. The pharmaceutical composition of claim 1, wherein the pharmaceutical composition further comprises a lipid nanoparticle (LNP).

16. The pharmaceutical composition of claim 1, which induces a humoral immune response or cellular immunity in a subject to the first immunogen.

17.-19. (canceled)

20. A method of inducing an immune response in a subject, the method comprising administering to the subject:
(a) a composition comprising an mRNA molecule encoding a first immunogen; and
(b) a composition comprising a double stranded DNA molecule encoding: (i) the first immunogen, (ii) a second immunogen having at least 90% amino acid sequence identity to the first immunogen, or (iii) a second immunogen that is recognized by an antigen binding site of an antibody or TCR, wherein the antigen binding site also recognizes the first immunogen.

21.-25. (canceled)

26. A kit comprising:
(a) an mRNA molecule encoding a first immunogen; and
(b) a double stranded DNA molecule encoding: (i) the first immunogen, (ii) a second immunogen having at least 90% amino acid sequence identity to the immunogen, or (iii) a second immunogen that is recognized by an antigen binding site of an antibody or TCR, wherein the antigen binding site also recognizes the first immunogen.

27. A method of making the pharmaceutical composition of claim 1, the method comprising: providing (a), providing (b), and admixing (a) with (b).

28. The pharmaceutical composition of claim 1, wherein the double stranded DNA molecule is circular.

29. The pharmaceutical composition of claim 1, wherein the double stranded DNA molecule comprises a first strand and a second strand, wherein the first strand comprises one or more chemically modified nucleobases, and the second strand is free of chemically modified nucleobases.

30. The pharmaceutical composition of claim 29, wherein the first strand is a sense strand and the second strand is an antisense strand.

31. The pharmaceutical composition of claim 29, wherein the chemically modified nucleobase comprises a uracil nucleobase.

32. The pharmaceutical composition of claim 31, wherein at least 1%, at least 5%, at least 10%, at least 15%, at least 20%, at least 25%, at least 50%, at least 75%, at least 90%, at least 95%, at least 96%, at least 97%, at least 98%, or at least 99% of thymine or uracil positions in the first strand of the dsDNA molecule comprise the uracil nucleobase.

33. The pharmaceutical composition of claim 31, wherein the uracil nucleobase is a canonical uracil nucleobase.
